(19) United States
(12) Patent Application Publication
(10) Pub. No.: US 2024/0142676 A1
(43) Pub. Date: May 2, 2024

(54) COMPOUND, COMPOSITION, CURED PRODUCT, OPTICALLY ANISOTROPIC BODY, OPTICAL ELEMENT, AND LIGHT GUIDE ELEMENT

(71) Applicant: FUJIFILM Corporation, Tokyo (JP)

(72) Inventors: Yuki FUKUSHIMA, Minami-Ashigara-shi (JP); Megumi OKUBO, Minami-Ashigara-shi (JP); Keisuke KODAMA, Minami-Ashigara-shi (JP); Shunya KATOH, Minami-Ashigara-shi (JP); Kiyoshi TAKEUCHI, Minami-Ashigara-shi (JP)

(73) Assignee: FUJIFILM Corporation, Tokyo (JP)

(21) Appl. No.: 18/392,996

(22) Filed: Dec. 21, 2023

Related U.S. Application Data
(63) Continuation of application No. PCT/JP2022/024994, filed on Jun. 22, 2022.

(30) Foreign Application Priority Data
Jun. 23, 2021 (JP) 2021-104474

(51) Int. Cl. G02B 5/18; C08F 20/30; G02B 5/30; G02B 6/27; G02F 1/1333

(52) U.S. Cl. CPC G02B 5/18; C08F 20/30; G02B 5/3016; G02B 6/2726; G02F 1/1333

(57) ABSTRACT

There is provided a compound represented by General Formula (I), a composition containing the compound represented by General Formula (I), a cured product, an optically anisotropic body, an optical element, and a light guide element. P¹ and P² each independently represent a hydrogen atom, —CN, —NCS, or a polymerizable group. However, at least one of P¹ or P² represents a polymerizable group. Sp¹ and Sp² each independently represent a specific group. A¹, A², A³, and A⁴ each independently represent an aromatic hydrocarbon ring group or aromatic heterocyclic group which may have a specific substituent. Z¹ and Z² each independently represent a specific linking group or a single bond. B¹ represents a group represented by a specific formula. n1 and n2 each independently represent an integer of 0 to 2.

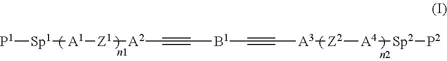

### CROSS REFERENCE TO RELATED APPLICATION

This is a continuation of International Application No. PCT/JP2022/024994 filed on Jun. 22, 2022, and claims priority from Japanese Patent Application No. 2021-104474 filed on Jun. 23, 2021, the entire disclosures of which are incorporated herein by reference.

### BACKGROUND OF THE INVENTION

### 1. Field of the Invention

The present invention relates to a compound, a composition, a cured product, an optically anisotropic body, an optical element, and a light guide element.

### 2. Description of the Related Art

A compound having liquid crystallinity (hereinafter, also referred to as a “liquid crystal compound”) and a composition having liquid crystallinity (hereinafter, also referred to as a “liquid crystal composition”) can be applied to various use applications.

For example, WO2020/022496A describes that diffracted light with high diffraction efficiency can be obtained at a large diffraction angle by an optical element including an optically anisotropic layer consisting of a cured product of a composition containing a liquid crystal compound. WO2020/022496A describes that good diffraction efficiency can be obtained by using a liquid crystal compound having a high refractive index anisotropy Δn (hereinafter, also simply referred to as “Δn”).

In addition, WO2018/034216A and JP2005-15406A discloses a liquid crystal compound having a high Δn. Further, WO2018/034216A discloses a reflective film obtained by curing a composition containing a liquid crystal compound having a high Δn.

### SUMMARY OF THE INVENTION

As described in WO2020/022496A, WO2018/034216A, and JP2005-15406A, a liquid crystal compound having a high Δn is useful for various use applications. In addition, for example, in a case of being mixed with another compound having liquid crystallinity, a compound having a high Δn can be used to form a liquid crystal composition having a high Δn even in a case where the compound itself does not have liquid crystallinity, which is useful in various use applications.

In addition, there is also a demand for improving the light resistance of such a compound.

An object of the present invention is to provide a compound having a high refractive index anisotropy Δn and having excellent light resistance and provide a composition, a cured product, an optically anisotropic body, an optical element, and a light guide element, each of which contains the compound.

As a result of intensive examination, the inventors of the present invention have found that the above object can be achieved by the following means.
<1> A compound represented by General Formula (I).

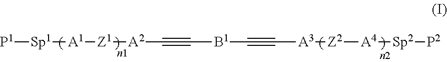

In General Formula (I),
P¹ and P² each independently represent a hydrogen atom, —CN, —NCS, or a polymerizable group,
provided that at least one of P¹ or P² represents a polymerizable group,
Sp¹ and Sp² each independently represent an alkylene group having 1 to 15 carbon atoms, where one or more methylene groups contained in the alkylene group may be each independently replaced with —O—, —S—, or —C(═O)—,
A¹, A², A³, and A⁴ each independently represent an aromatic hydrocarbon ring group or aromatic heterocyclic group which may have a substituent L1,
in a case where a plurality of A¹'s are present, the plurality of A¹'s may be the same or different from each other,
in a case where a plurality of A⁴'s are present, the plurality of A⁴'s may be the same or different from each other,
Z¹ and Z² each independently represent —O—, —S—, —CHRCHR—, —OCHR—, —CHRO—, —CO—, —SO—, —SO₂—, —COO—, —OCO—, —CO—S—, —S—CO—, —O—CO—O—, —CO—NR—, —NR—CO—, —SCHR—, —CHRS—, —SO—CHR—, —CHR—SO—, —SO₂—CHR—, —CHR—SO₂—, —CF₂O—, —OCF₂—, —CF₂S—, —SCF₂—, —OCHRCHRO—, —SCHRCHRS—, —SO—CHRCHR—SO—, —SO₂—CHRCHR—SO₂—, —CH═CH—COO—, —CH═CH—OCO—, —COO—CH═CH—, —OCO—CH═CH—, —COO—CHRCHR—, —OCO—CHRCHR—, —CHRCHR—COO—, —CHRCHR—OCO—, —COO—CHR—, —OCO—CHR—, —CHR—COO—, —CHR—OCO—, —CR═CR—, —CR═N—, —N═CR—, —N═N—, —CR═N—N═CR—, —CF═CF—, —C≡C—, or a single bond,
R represents a hydrogen atom or an alkyl group having 1 to 10 carbon atoms,
in a case where a plurality of R's are present, the plurality of R's may be the same or different from each other,
in a case where a plurality of Z¹'s are present, the plurality of Z¹'s may be the same or different from each other,
in a case where a plurality of Z²'s are present, the plurality of Z²'s may be the same or different from each other, and
B¹ represents a group represented by any one of General Formulae (B-1) to (B-4).

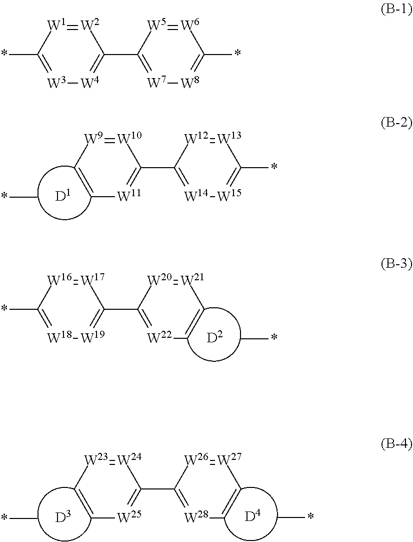

In General Formulae (B-1) to (B-4),
W¹ to W²⁸ each independently represent CR¹ or N, where R¹ represents a hydrogen atom, an alkyl group having 1 to 10 carbon atoms, an alkoxy group having 1 to 10 carbon atoms, an alkylamino group having 1 to 10 carbon atoms, an alkylthio group having 1 to 10 carbon atoms, an alkanoyl group having 1 to 10 carbon atoms, an alkanoyloxy group having 1 to 10 carbon atoms, an alkanoylamino group having 1 to 10 carbon atoms, an alkanoylthio group having 1 to 10 carbon atoms, an alkyloxycarbonyl group having 2 to 10 carbon atoms, an alkylaminocarbonyl group having 2 to 10 carbon atoms, an alkylthiocarbonyl group having 2 to 10 carbon atoms, a hydroxy group, an amino group, a mercapto group, a carboxy group, a sulfo group, an amide group, a cyano group, a nitro group, a halogen atom, a sulfhydryl group, an aldehyde group, or a polymerizable group,
provided that in a case where the group has —CH₂—, at least one —CH₂— contained in the group may be replaced with —O—, —CO—, —CH═CH—, or —C≡C—, and in a case where the group has a hydrogen atom, at least one hydrogen atom contained in the group may be replaced with at least one selected from the group consisting of a fluorine atom and a polymerizable group,
in General Formula (B-1), at least one of W² or W⁵ represents CR¹, where R¹ represents a group other than a hydrogen atom,
at least one of W⁴ or W⁷ represents CR¹, where R¹ represents a group other than a hydrogen atom,
in General Formula (B-2), at least one of W¹⁰ or W¹² represents CR¹, where R¹ represents a group other than a hydrogen atom,
at least one of W¹¹ or W¹⁴ represents CR¹, where R¹ represents a group other than a hydrogen atom,
in General Formula (B-3), at least one of W¹⁷ or W²⁰ represents CR¹, where R¹ represents a group other than a hydrogen atom,
at least one of W¹⁹ or W²² represents CR¹, where R¹ represents a group other than a hydrogen atom,
in General Formula (B-4), at least one of W²⁴ or W²⁶ represents CR¹, where R¹ represents a group other than a hydrogen atom,
at least one of W²⁵ or W²⁸ represents CR¹, where R¹ represents a group other than a hydrogen atom,
D¹, D², D³, and D⁴ each independently represent an aromatic hydrocarbon ring group or aromatic heterocyclic group which may have a substituent L2,
* represents a bonding position,
the substituent L1 and the substituent L2 each independently represent an alkyl group having 1 to 10 carbon atoms, an alkoxy group having 1 to 10 carbon atoms, an alkylamino group having 1 to 10 carbon atoms, an alkylthio group having 1 to 10 carbon atoms, an alkanoyl group having 1 to 10 carbon atoms, an alkanoyloxy group having 1 to 10 carbon atoms, an alkanoylamino group having 1 to 10 carbon atoms, an alkanoylthio group having 1 to 10 carbon atoms, an alkyloxycarbonyl group having 2 to 10 carbon atoms, an alkylaminocarbonyl group having 2 to 10 carbon atoms, an alkylthiocarbonyl group having 2 to 10 carbon atoms, a hydroxy group, an amino group, a mercapto group, a carboxy group, a sulfo group, an amide group, a cyano group, a nitro group, a halogen atom, a sulfhydryl group, an aldehyde group, or a polymerizable group,
provided that in a case where the group has —CH₂—, at least one —CH₂— contained in the group may be replaced with —O—, —CO—, —CH═CH—, or —C≡C—, and in a case where the group has a hydrogen atom, at least one hydrogen atom contained in the group may be replaced with at least one selected from the group consisting of a fluorine atom and a polymerizable group, and
n1 and n2 each independently represent an integer of 0 to 2.
<2> The compound according to <1 >, in which n1 and n2 in General Formula (I) represent 0.
<3> The compound according to <1> or <2>, in which B¹ in General Formula (I) is represented by General Formula (B-1).
<4> The compound according to any one of <1> to <3>, in which A¹, A², A³, and A⁴ in General Formula (I) are each independently a group represented by any one of General Formulae (A-1) to (A-3), which may have the substituent L1.

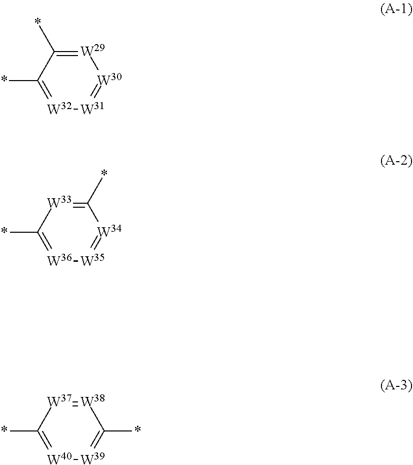

In General Formulae (A-1) to (A-3),
W²⁹ to W⁴⁰ each independently represent CR¹ or N, where R¹ represents a hydrogen atom or the substituent L1, and
* represents a bonding position.
<5> The compound according to any one of <1> to <4>, in which in General Formula (I), Sp¹ represents a group represented by General Formula (II), and Sp² represents a group represented by General Formula (III).

*—S—W⁴¹—** (II)

*—S—W⁴²—** (III)

In General Formulae (II) and (III),
W⁴¹ and W⁴² each independently represent an alkylene group having 1 to 15 carbon atoms, where one or more methylene groups contained in the alkylene group may be each independently replaced with —O—, —S—, or —C(═O)—,
*'s each represent a bonding position to A¹, A², A³, or A⁴, which is directly linked to Sp¹ or Sp², and
**'s each represent a bonding position to P¹ or P².
<6> The compound according to any one of <1> to <5>, in which the compound has liquid crystallinity.
<7> A composition comprising the compound according to any one of <1> to <6>.
<8> The composition according to <7>, further comprising a polymerization initiator.
<9> The composition according to <7> or <8>, further comprising a chiral agent.
<10> The composition according to any one of <7> to <9>, in which the composition has liquid crystallinity.
<11> The composition according to any one of <7> to <10>, in which the composition is used for forming an optically anisotropic layer.
<12> A cured product that is obtained by curing the composition according to any one of <7> to <10>.
<13> An optically anisotropic body that is obtained by curing the composition according to any one of <7> to <10>.
<14> An optical element comprising:
an optically anisotropic layer formed from the composition according to any one of <7> to <10>,
in which the optically anisotropic layer has an alignment pattern, and
the alignment pattern is an alignment pattern in which an orientation of an optical axis, derived from a compound contained in the composition, continuously changes rotationally along at least one in-plane direction.
<15> A light guide element comprising the optical element according to <14> and a light guide plate.

According to the present invention, it is possible to provide a compound having a high refractive index anisotropy Δn and having excellent light resistance and provide a composition, a cured product, an optically anisotropic body, an optical element, and a light guide element, each of which contains the compound.

### DESCRIPTION OF THE PREFERRED EMBODIMENTS

Hereinafter, embodiments of the present invention will be specifically described, but the present invention is not limited thereto. In the present specification, in a case where numerical values represent a value of physical properties, a value of characteristics, and the like, the description of “(numerical value 1) to (numerical value 2)” means “(numerical value 1) or more and (numerical value 2) or less”. In addition, in the present specification, the description of “(meth)acrylate” means “at least any one of acrylate or methacrylate”. The same applies to “(meth)acrylic acid”, “(meth)acryloyl”, “(meth)acrylamide”, “(meth)acryloyloxy”.

### Compound Represented by General Formula (I)

Hereinafter, a compound represented by General Formula (I) will be described in detail.

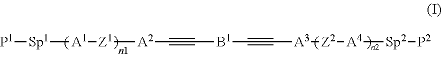

In General Formula (I),
P¹ and P² each independently represent a hydrogen atom, —CN, —NCS, or a polymerizable group,
provided that at least one of P¹ or P² represents a polymerizable group,
Sp¹ and Sp² each independently represent an alkylene group having 1 to 15 carbon atoms, where one or more methylene groups contained in the alkylene group may be each independently replaced with —O—, —S—, or —C(═O)—,
A¹, A², A³, and A⁴ each independently represent an aromatic hydrocarbon ring group or aromatic heterocyclic group which may have a substituent L1,
in a case where a plurality of A¹'s are present, the plurality of A¹'s may be the same or different from each other, and in a case where a plurality of A⁴'s are present, the plurality of A⁴'s may be the same or different from each other.

Z¹ and Z² each independently represent —O—, —S—, —CHRCHR—, —OCHR—, —CHRO—, —CO—, —SO—, —SO₂—, —COO—, —OCO—, —CO—S—, —S—CO—, —O—CO—O—, —CO—NR—, —NR—CO—, —SCHR—, —CHRS—, —SO—CHR—, —CHR—SO—, —SO₂—CHR—, —CHR—SO₂—, —CF₂O—, —OCF₂—, —CF₂S—, —SCF₂—, —OCHRCHRO—, —SCHRCHRS—, —SO—CHRCHR—SO—, —SO₂—CHRCHR—SO₂, —CH═CH—COO—, —CH═CH—OCO—, —COO—CH═CH—, —OCO—CH═CH—, —COO—CHRCHR—, —OCO—CHRCHR—, —CHRCHR—COO—, —CHRCHR—OCO—, —COO—CHR—, —OCO—CHR—, —CHR—COO—, —CHR—OCO—, —CR═CR—, —CR═N—, —N═CR—, —N═N—, —CR═N—N═CR—, —CF═CF—, —C≡C—, or a single bond.

R represents a hydrogen atom or an alkyl group having 1 to 10 carbon atoms.

In a case where a plurality of R's are present, the plurality of R's may be the same or different from each other.

In a case where a plurality of Z¹'s are present, the plurality of Z¹'s may be the same or different from each other.

In a case where a plurality of Z²'s are present, the plurality of Z²'s may be the same or different from each other.

B¹ represents a group represented by any one of General Formulae (B-1) to (B-4).

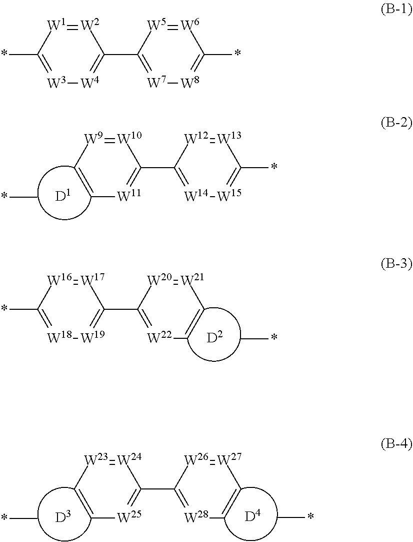

In General Formulae (B-1) to (B-4),
W¹ to W²⁸ each independently represent CR¹ or N, where R¹ represents a hydrogen atom, an alkyl group having 1 to 10 carbon atoms, an alkoxy group having 1 to 10 carbon atoms, an alkylamino group having 1 to 10 carbon atoms, an alkylthio group having 1 to 10 carbon atoms, an alkanoyl group having 1 to 10 carbon atoms, an alkanoyloxy group having 1 to 10 carbon atoms, an alkanoylamino group having 1 to 10 carbon atoms, an alkanoylthio group having 1 to 10 carbon atoms, an alkyloxycarbonyl group having 2 to 10 carbon atoms, an alkylaminocarbonyl group having 2 to 10 carbon atoms, an alkylthiocarbonyl group having 2 to 10 carbon atoms, a hydroxy group, an amino group, a mercapto group, a carboxy group, a sulfo group, an amide group, a cyano group, a nitro group, a halogen atom, a sulfhydryl group, an aldehyde group, or a polymerizable group,
provided that in a case where the group has —CH₂—, at least one —CH₂— contained in the group may be replaced with —O—, —CO—, —CH═CH—, or —C≡C—, and in a case where the group has a hydrogen atom, at least one hydrogen atom contained in the group may be replaced with at least one selected from the group consisting of a fluorine atom and a polymerizable group.

In General Formula (B-1), at least one of W² or W⁵ represents CR¹, where R¹ represents a group other than a hydrogen atom.

At least one of W⁴ or W⁷ represents CR¹, where R¹ represents a group other than a hydrogen atom.

In General Formula (B-2), at least one of W¹⁰ or W¹² represents CR¹, where R¹ represents a group other than a hydrogen atom.

At least one of W¹¹ or W¹⁴ represents CR¹, where R¹ represents a group other than a hydrogen atom.

In General Formula (B-3), at least one of W¹⁷ or W²⁰ represents CR¹, where R¹ represents a group other than a hydrogen atom.

At least one of W¹⁹ or W²² represents CR¹, where R¹ represents a group other than a hydrogen atom.

In General Formula (B-4), at least one of W²⁴ or W²⁶ represents CR¹, where R¹ represents a group other than a hydrogen atom.

At least one of W²⁵ or W²⁸ represents CR¹, where R¹ represents a group other than a hydrogen atom.

D¹, D², D³, and D⁴ each independently represent an aromatic hydrocarbon ring group or aromatic heterocyclic group which may have a substituent L2.

* represents a bonding position.

The substituent L1 and the substituent L2 each independently represent an alkyl group having 1 to 10 carbon atoms, an alkoxy group having 1 to 10 carbon atoms, an alkylamino group having 1 to 10 carbon atoms, an alkylthio group having 1 to 10 carbon atoms, an alkanoyl group having 1 to 10 carbon atoms, an alkanoyloxy group having 1 to 10 carbon atoms, an alkanoylamino group having 1 to 10 carbon atoms, an alkanoylthio group having 1 to 10 carbon atoms, an alkyloxycarbonyl group having 2 to 10 carbon atoms, an alkylaminocarbonyl group having 2 to 10 carbon atoms, an alkylthiocarbonyl group having 2 to 10 carbon atoms, a hydroxy group, an amino group, a mercapto group, a carboxy group, a sulfo group, an amide group, a cyano group, a nitro group, a halogen atom, a sulfhydryl group, an aldehyde group, or a polymerizable group. However, in a case where the group has —CH₂—, at least one —CH₂— contained in the group may be replaced with —O—, —CO—, —CH═CH—, or —C≡C—. In addition, in a case where the group has a hydrogen atom, at least one hydrogen atom contained in the group may be replaced with at least one selected from the group consisting of a fluorine atom and a polymerizable group.

n1 and n2 each independently represent an integer of 0 to 2.

P¹ and P² each independently represent a hydrogen atom, —CN, —NCS, or a polymerizable group.

However, at least one of P¹ or P² represents a polymerizable group.

The kind of the polymerizable group is not particularly limited, and examples thereof include a well-known polymerizable group. From the viewpoint of reactivity, a functional group capable of an addition polymerization reaction is preferable, and a polymerizable ethylenically unsaturated group or a ring polymerizable group is more preferable. Examples of the polymerizable group include a (meth)acryloyloxy group, a vinyl group, a maleimide group, a styryl group, an allyl group, an epoxy group, an oxetane group, and a group including the above-described group. A hydrogen atom in each of the above groups may be substituted with another substituent such as a halogen atom.

Suitable specific examples of the polymerizable group include groups represented by General Formulae (P-1) to (P-19). In the following formulae, * represents a bonding position, Me represents a methyl group, and Et represents an ethyl group.

The polymerizable group is preferably a (meth)acryloyloxy group.

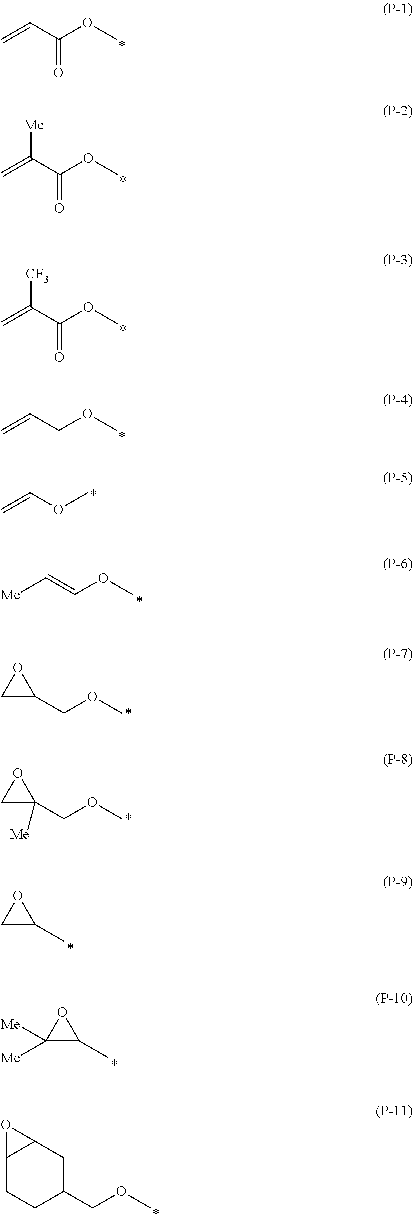

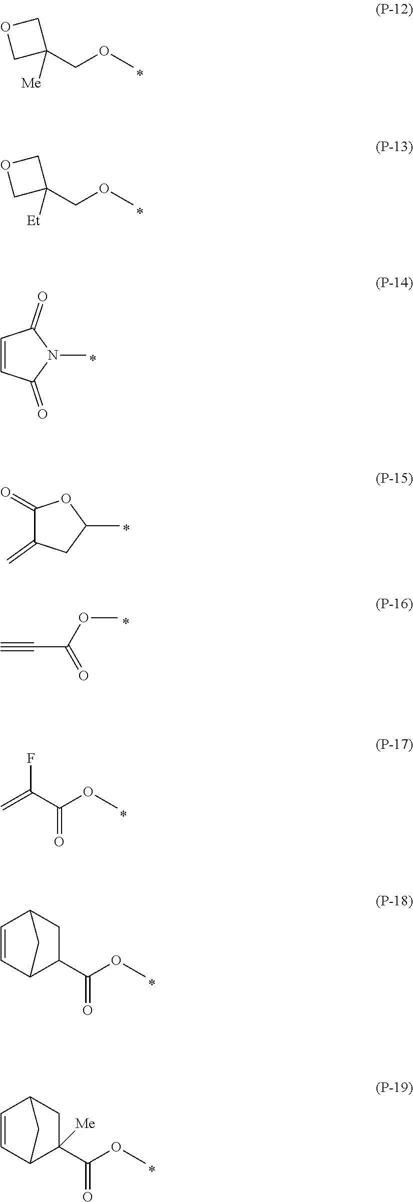

Among these, from the viewpoint that the reactivity is more excellent, both P¹ and P² are preferably polymerizable groups.

Sp¹ and Sp² each independently represent an alkylene group having 1 to 15 carbon atoms, where one or more methylene groups contained in the alkylene group may be each independently replaced with —O—, —S—, or —C(═O)—,

The alkylene group having 1 to 15 carbon atoms may be linear or branched, and it is preferably a linear alkylene group having 1 to 10 carbon atoms and more preferably a linear alkylene group having 1 to 5 carbon atoms.

It is preferable that in General Formula (I), Sp¹ represents a group represented by General Formula (II), and Sp² represents a group represented by General Formula (III). Since the group represented by General Formula (II) and the group represented by General Formula (III) contain a sulfur atom, it is possible to increase the refractive index anisotropy Δn of the compound represented by General Formula (I), which is preferable.

*—S—W⁴¹—** (II)

*—S—W⁴²—** (III)

In General Formulae (II) and (III),
W⁴¹ and W⁴² each independently represent an alkylene group having 1 to 15 carbon atoms, where one or more methylene groups contained in the alkylene group may be each independently replaced with —O—, —S—, or —C(═O)—,
*'s each represent a bonding position to A¹, A², A³, or A⁴, which is directly linked to Sp¹ or Sp², and
**'s each represent a bonding position to P¹ or P².

The alkylene group having 1 to 15 carbon atoms as W⁴¹ and W⁴² may be linear or branched, and it is preferably a linear alkylene group having 1 to 10 carbon atoms and more preferably a linear alkylene group having 1 to 5 carbon atoms.

A¹, A², A³, and A⁴ each independently represent an aromatic hydrocarbon ring group or aromatic heterocyclic group which may have a substituent L1. In a case where a plurality of A¹'s are present, the plurality of A¹'s may be the same or different from each other, and in a case where a plurality of A⁴'s are present, the plurality of A⁴'s may be the same as or different from each other.

The aromatic hydrocarbon ring group may have a monocyclic structure or may have a polycyclic structure. The aromatic hydrocarbon ring group is not particularly limited; however, it is preferably an arylene group, more preferably an arylene group having 6 to 20 carbon atoms, still more preferably an arylene group having 6 to 10 carbon atoms, and particularly preferably a phenylene group or a naphthyl group.

The aromatic heterocyclic group may have a monocyclic structure or may have a polycyclic structure. The aromatic heterocyclic group is not particularly limited; however, it is preferably a heteroarylene group, more preferably a heteroarylene group having 3 to 20 carbon atoms, and still more preferably a heteroarylene group having 3 to 10 carbon atoms. The heteroatom contained in the heteroarylene group is preferably at least one selected from the group consisting of a nitrogen atom, an oxygen atom, and a sulfur atom.

The aromatic hydrocarbon ring group and aromatic heterocyclic group may have a substituent L1. The substituent L1 will be described later. The substituent L1 may be further substituted with a substituent L1. In addition, the number of substituents L1 is not particularly limited, and the aromatic hydrocarbon ring group and the aromatic heterocyclic group may have one substituent L1 or may have a plurality of substituents L1.

It is preferable that A¹, A², A³, and A⁴ in General Formula (I) are each independently a group represented by any one of General Formulae (A-1) to (A-3), which may have the substituent L1. This is preferable since the solubility of the compound represented by General Formula (I) can be further improved.

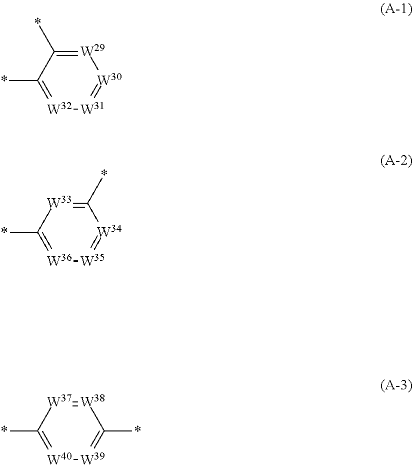

In General Formulae (A-1) to (A-3),
W²⁹ to W⁴⁰ each independently represent CR¹ or N, where R¹ represents a hydrogen atom or the substituent L1, and
* represents a bonding position.

In General Formula (A-1), in a case where a plurality of R¹'s are present, the plurality of R¹'s may be the same or different from each other.

In General Formula (A-2), in a case where a plurality of R¹'s are present, the plurality of R¹'s may be the same or different from each other.

In General Formula (A-3),
in a case where a plurality of R¹'s are present, the plurality of R¹'s may be the same or different from each other.

It is preferable that A¹, A², A³, and A⁴ each independently represent a group represented General Formula (A-3), which may have the substituent L1.

Examples of the preferred aspect thereof include an aspect in which A¹, A², A³, and A⁴ each independently represent the group represented by General Formula (A-3), which may have the substituent L1, and W³⁷ to W⁴⁰ each independently represent CR¹, where R¹ represents a hydrogen atom.

Z¹ and Z² each independently represent —O—, —S—, —CHRCHR—, —OCHR—, —CHRO—, —CO—, —SO—, —SO₂—, —COO—, —OCO—, —CO—S—, —S—CO—, —O—CO—O—, —CO—NR—, —NR—CO—, —SCHR—, —CHRS—, —SO—CHR—, —CHR—SO—, —SO₂—CHR—, —CHR—SO₂—, —CF₂O—, —OCF₂—, —CF₂S—, —SCF₂—, —OCHRCHRO—, —SCHRCHRS—, —SO—CHRCHR—SO—, —SO₂—CHRCHR—SO₂—, —CH═CH—COO—, —CH═CH—OCO—, —COO—CH═CH—, —OCO—CH═CH—, —COO—CHRCHR—, —OCO—CHRCHR—, —CHRCHR—COO—, —CHRCHR—OCO—, —COO—CHR—, —OCO—CHR—, —CHR—COO—, —CHR—OCO—, —CR═CR—, —CR═N—, —N═CR—, —N═N—, —CR═N—N═CR—, —CF═CF—, —C≡C—, or a single bond.

R represents a hydrogen atom or an alkyl group having 1 to 10 carbon atoms.

In a case where a plurality of R's are present, the plurality of R's may be the same or different from each other.

In a case where a plurality of Z¹'s are present, the plurality of Z¹'s may be the same or different from each other.

In a case where a plurality of Z²'s are present, the plurality of Z²'s may be the same or different from each other.

Z¹ and Z² are each independently preferably —CHRCHR—, —OCHR—, —CHRO—, —COO—, —OCO—, —CO—NH—, —NH—CO—, —C≡C—, or a single bond, and still more preferably —CHRCHR—, —OCHR—, or —CHRO—.

R represents a hydrogen atom or an alkyl group having 1 to 10 carbon atoms, preferably represents a hydrogen atom or an alkyl group having 1 to 6 carbon atoms, more preferably represents a hydrogen atom or an alkyl group having 1 to 4 carbon atoms, and still more preferably represent a hydrogen atom.

B¹ represents a group represented by any one of General Formulae (B-1) to (B-4).

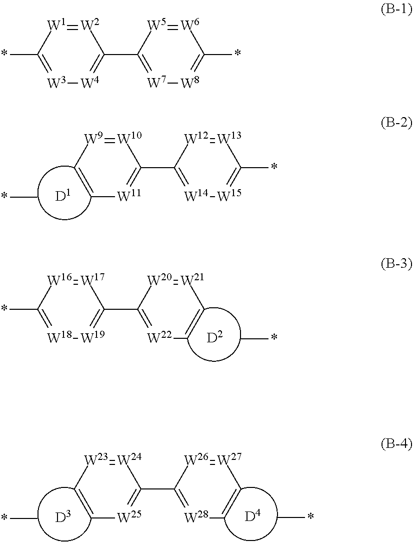

In General Formulae (B-1) to (B-4),
W¹ to W²⁸ each independently represent CR¹ or N, where R¹ represents a hydrogen atom, an alkyl group having 1 to 10 carbon atoms, an alkoxy group having 1 to 10 carbon atoms, an alkylamino group having 1 to 10 carbon atoms, an alkylthio group having 1 to 10 carbon atoms, an alkanoyl group having 1 to 10 carbon atoms, an alkanoyloxy group having 1 to 10 carbon atoms, an alkanoylamino group having 1 to 10 carbon atoms, an alkanoylthio group having 1 to 10 carbon atoms, an alkyloxycarbonyl group having 2 to 10 carbon atoms, an alkylaminocarbonyl group having 2 to 10 carbon atoms, an alkylthiocarbonyl group having 2 to 10 carbon atoms, a hydroxy group, an amino group, a mercapto group, a carboxy group, a sulfo group, an amide group, a cyano group, a nitro group, a halogen atom, a sulfhydryl group, an aldehyde group, or a polymerizable group, provided that in a case where the group has —CH₂—, at least one —CH₂— contained in the group may be replaced with —O—, —CO—, —CH═CH—, or —C≡C—, and in a case where the group has a hydrogen atom, at least one hydrogen atom contained in the group may be replaced with at least one selected from the group consisting of a fluorine atom and a polymerizable group. Examples of the polymerizable group include the same ones as the groups described as the polymerizable group as P¹ or P² described above, and the same applies to the preferred range thereof.

In General Formula (B-1), at least one of W² or W⁵ represents CR¹, where R¹ represents a group other than a hydrogen atom.

At least one of W⁴ or W⁷ represents CR¹, where R¹ represents a group other than a hydrogen atom.

In General Formula (B-1), in a case where a plurality of R¹'s are present, the plurality of R¹'s may be the same or different from each other.

In General Formula (B-2), at least one of W¹⁰ or W¹² represents CR¹, where R¹ represents a group other than a hydrogen atom.

At least one of W¹¹ or W¹⁴ represents CR¹, where R¹ represents a group other than a hydrogen atom.

In General Formula (B-2), in a case where a plurality of R¹'s are present, the plurality of R¹'s may be the same or different from each other.

In General Formula (B-3), at least one of W¹⁷ or W²⁰ represents CR¹, where R¹ represents a group other than a hydrogen atom.

At least one of W¹⁹ or W²² represents CR¹, where R¹ represents a group other than a hydrogen atom.

In General Formula (B-3), in a case where a plurality of R¹'s are present, the plurality of R¹'s may be the same or different from each other.

In General Formula (B-4), at least one of W²⁴ or W²⁶ represents CR¹, where R¹ represents a group other than a hydrogen atom.

At least one of W²⁵ or W²⁸ represents CR¹, where R¹ represents a group other than a hydrogen atom.

In General Formula (B-4), in a case where a plurality of R¹'s are present, the plurality of R¹'s may be the same or different from each other.

Examples of the polymerizable group as R¹ include the same ones as the groups described as the polymerizable group as P¹ or P² described above, and the same applies to the preferred range thereof.

R¹ is preferably a hydrogen atom, an alkyl group having 1 to 10 carbon atoms, an alkoxy group having 1 to 10 carbon atoms, an alkanoyl group having 1 to 10 carbon atoms, an alkanoyloxy group having 1 to 10 carbon atoms, an alkyloxycarbonyl group having 2 to 10 carbon atoms, a hydroxy group, a carboxy group, a cyano group, a nitro group, a trifluoromethyl group, or a halogen atom.

R¹ is more preferably a hydrogen atom, an alkyl group having 1 to 10 carbon atoms, an alkoxy group having 1 to 10 carbon atoms, an alkanoyl group having 2 to 10 carbon atoms, an alkanoyloxy group having 2 to 10 carbon atoms, an alkyloxycarbonyl group having 2 to 10 carbon atoms, a trifluoromethyl group, or a halogen atom.

The number of carbon atoms of each of the alkyl group and the alkoxy group is more preferably 1 to 5 and still more preferably 1 to 3. The number of carbon atoms of each of the alkanoyl group and the alkanoyloxy group is more preferably 2 to 5 and still more preferably 2 or 3. The number of carbon atoms of the alkyloxycarbonyl group is more preferably 2 to 5 and still more preferably 2 or 3.

As described above, in a case where each group as R¹ has —CH₂—, at least one —CH₂— contained in the group may be replaced with —O—, —CO—, —CH═CH—, or —C≡C—.

In a case where each group as R¹ has a hydrogen atom, at least one hydrogen atom contained in the group may be replaced with at least one selected from the group consisting of a fluorine atom and a polymerizable group.

D¹, D², D³, and D⁴ each independently represent an aromatic hydrocarbon ring group or aromatic heterocyclic group which may have a substituent L2.

* represents a bonding position.

The aromatic hydrocarbon ring group may have a monocyclic structure or may have a polycyclic structure. Specific examples of the ring constituting the aromatic hydrocarbon group include a benzene ring, a biphenyl ring, a naphthalene ring, an anthracene ring, a phenanthrene ring, and a fluorene ring. Among these, a benzene ring is preferable.

The aromatic heterocyclic group may have a monocyclic structure or may have a polycyclic structure. Specific examples of the ring constituting the aromatic heterocyclic group include a furan ring, a thiophene ring, a pyrrole ring, an oxazole ring, an isoxazole ring, an oxadiazole ring, a thiazole ring, an isothiazole ring, a thiadiazole ring, an imidazole ring, a pyrazole ring, a triazole ring, a furazan ring, a tetrazole ring, a pyridine ring, a pyridazine ring, a pyrimidine ring, a pyrazine ring, a triazine ring, a tetrazine ring, and a benzothiazole ring.

The aromatic hydrocarbon ring group and aromatic heterocyclic group may have a substituent L2. The substituent L2 will be described later.

In General Formula (B-2), D¹ is fused with a ring containing W⁹ to W¹¹.

In General Formula (B-3), D² is fused with a ring containing W²⁰ to W²².

In General Formula (B-4), D³ is fused with a ring containing W²³ to W²⁵, and D⁴ is fused with a ring containing W²⁶ to W²⁸.

The substituent L1 and the substituent L2 each independently represent an alkyl group having 1 to 10 carbon atoms, an alkoxy group having 1 to 10 carbon atoms, an alkylamino group having 1 to 10 carbon atoms, an alkylthio group having 1 to 10 carbon atoms, an alkanoyl group having 1 to 10 carbon atoms, an alkanoyloxy group having 1 to 10 carbon atoms, an alkanoylamino group having 1 to 10 carbon atoms, an alkanoylthio group having 1 to 10 carbon atoms, an alkyloxycarbonyl group having 2 to 10 carbon atoms, an alkylaminocarbonyl group having 2 to 10 carbon atoms, an alkylthiocarbonyl group having 2 to 10 carbon atoms, a hydroxy group, an amino group, a mercapto group, a carboxy group, a sulfo group, an amide group, a cyano group, a nitro group, a halogen atom, a sulfhydryl group, an aldehyde group, or a polymerizable group.

However, in a case where the group has —CH₂—, at least one —CH₂— contained in the group may be replaced with —O—, —CO—, —CH═CH—, or —C≡C—. In addition, in a case where the group has a hydrogen atom, at least one hydrogen atom contained in the group may be replaced with at least one selected from the group consisting of a fluorine atom and a polymerizable group. Examples of the polymerizable group include the same ones as the groups described as the polymerizable group as P¹ or P² described above, and the same applies to the preferred range thereof.

Examples of the polymerizable group as substituent L1 and the substituent L2 include the same ones as the groups described as the polymerizable group as P¹ or P² described above, and the same applies to the preferred range thereof.

The substituent L1 and the substituent L2 are each independently preferably an alkyl group having 1 to 10 carbon atoms, an alkoxy group having 1 to 10 carbon atoms, an alkanoyl group having 1 to 10 carbon atoms, an alkanoyloxy group having 1 to 10 carbon atoms, an alkyloxycarbonyl group having 2 to 10 carbon atoms, a hydroxy group, a carboxy group, a cyano group, a nitro group, a trifluoromethyl group, or a halogen atom.

The substituent L1 and the substituent L2 are each independently more preferably an alkyl group having 1 to 10 carbon atoms, an alkoxy group having 1 to 10 carbon atoms, an alkanoyl group having 2 to 10 carbon atoms, an alkanoyloxy group having 2 to 10 carbon atoms, an alkyloxycarbonyl group having 2 to 10 carbon atoms, a trifluoromethyl group, or a halogen atom.

The substituent L1 and the substituent L2 are each independently still more preferably an alkyl group having 1 to 6 carbon atoms, an alkoxy group having 1 to 6 carbon atoms, an alkanoyl group having 2 to 6 carbon atoms, an alkanoyloxy group having 2 to 6 carbon atoms, an alkyloxycarbonyl group having 2 to 6 carbon atoms, or a trifluoromethyl group.

As described above, in a case where each group as the substituent L1 and the substituent L2 has —CH₂—, at least one —CH₂— contained in the group may be replaced with —O—, —CO—, —CH═CH—, or —C≡C—. In addition, in a case where each group as substituent L1 and the substituent L2 has a hydrogen atom, at least one hydrogen atom contained in the group may be replaced with at least one selected from the group consisting of a fluorine atom and a polymerizable group.

It is preferable that B¹ is a group represented by General Formula (B-1). This is preferable since the solubility of the compound represented by General Formula (I) can be further improved.

Examples of the preferred aspect thereof include an aspect in which in the group represented by General Formula (B-1), W¹ to W⁸ each independently represent CR¹ or N, R¹ in W² represents an alkyl group having 1 to 10 carbon atoms, and R¹ in W⁷ represents an alkyl group having 1 to 10 carbon atoms.

Examples of the preferred aspect thereof include an aspect in which in the group represented by General Formula (B-1), W¹ to W³ and W⁶ to W⁸ each independently represent CR¹ or N, W⁴ and W⁵ represent N, R¹ in W² represents an alkyl group having 1 to 10 carbon atoms, and R¹ in W⁷ represents an alkyl group having 1 to 10 carbon atoms.

Examples of the preferred aspect thereof include an aspect in which in the group represented by General Formula (B-1), W¹ to W⁸ each independently represent CR¹ or N, R¹ in W² and W⁴ each independently represent an alkyl group having 1 to 10 carbon atoms, and R¹ in W⁵ and W⁷ each independently represent an alkyl group having 1 to 10 carbon atoms.

n1 and n2 each independently represent an integer of 0 to 2. n1 and n2 each independently represent preferably 0 or 1, and more preferably represent 0. This is preferable since the solubility of the compound represented by General Formula (I) can be further improved.

Specific examples of the compound represented by General Formula (I) are shown below, which are not limited thereto. Me represents a methyl group, Et represents an ethyl group, and t-Bu represents a t-butyl group.

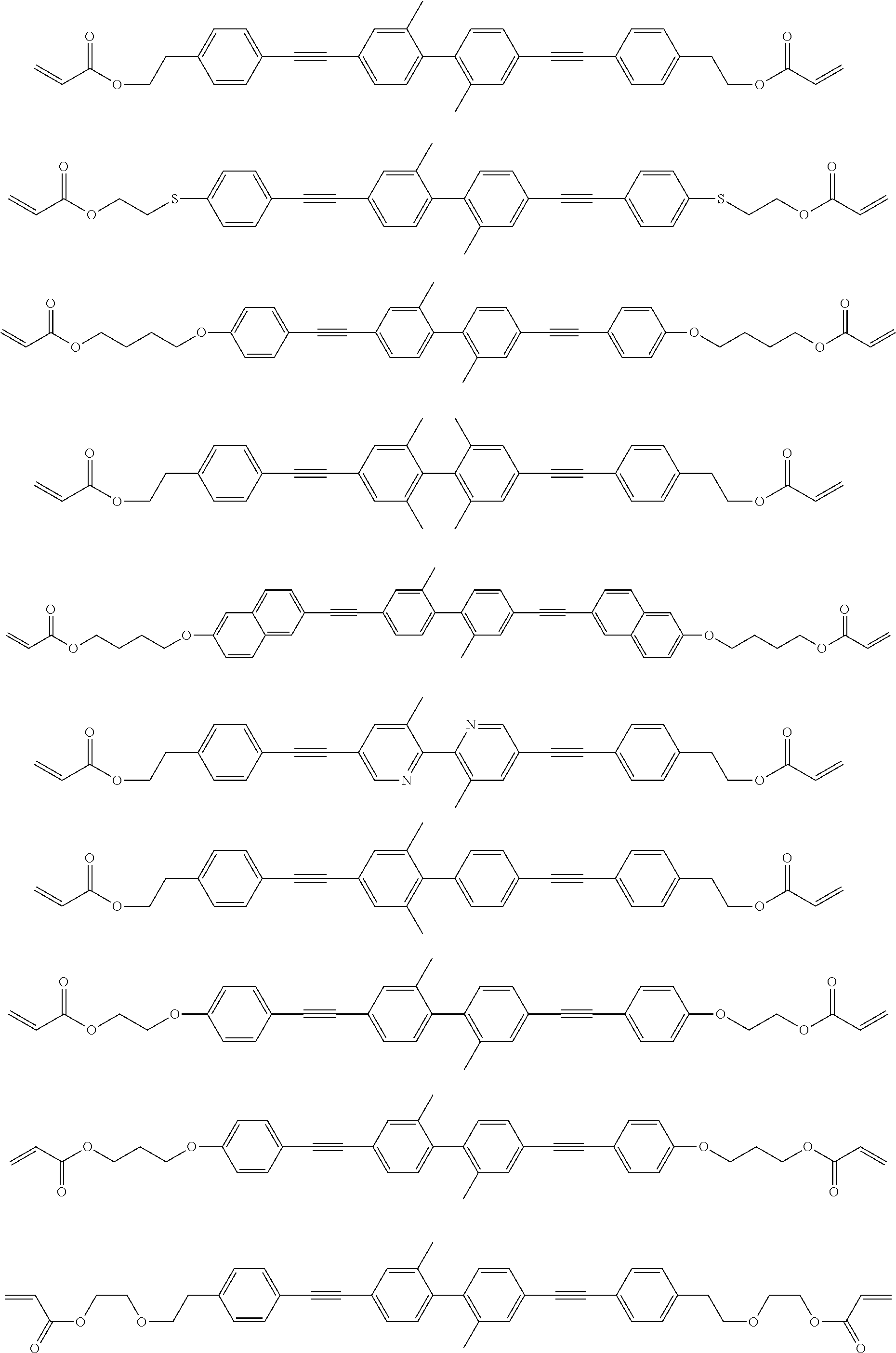

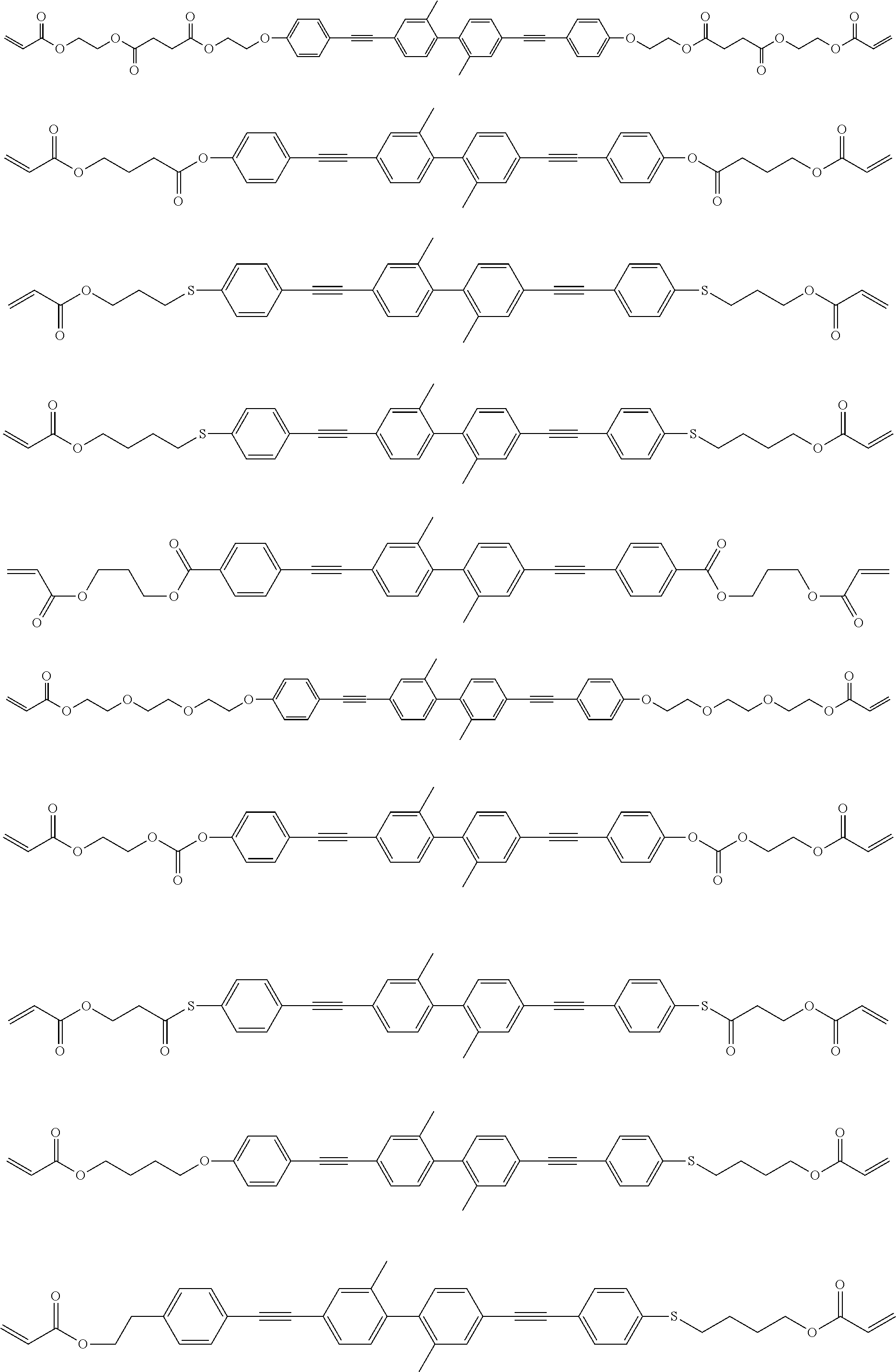

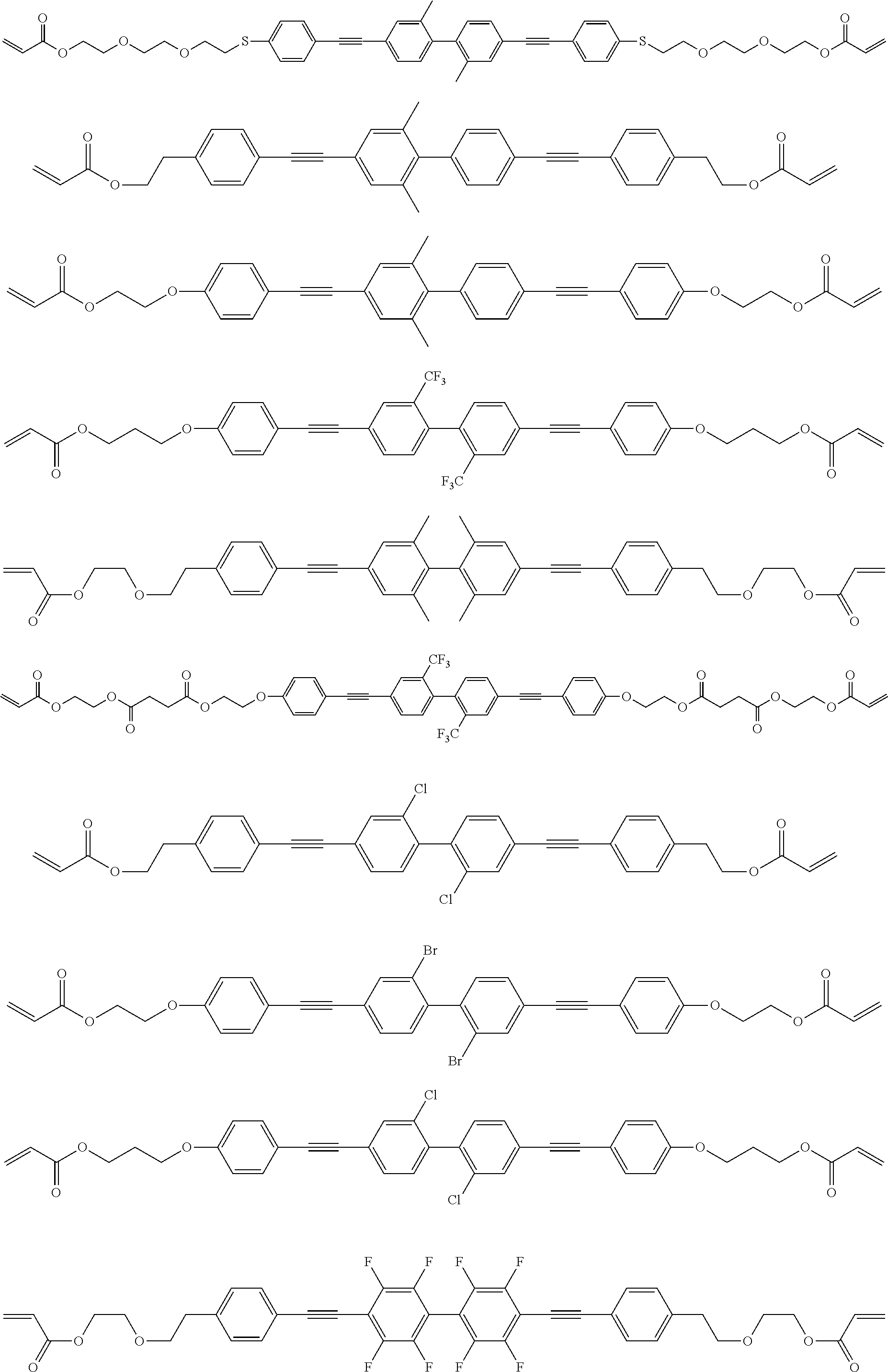

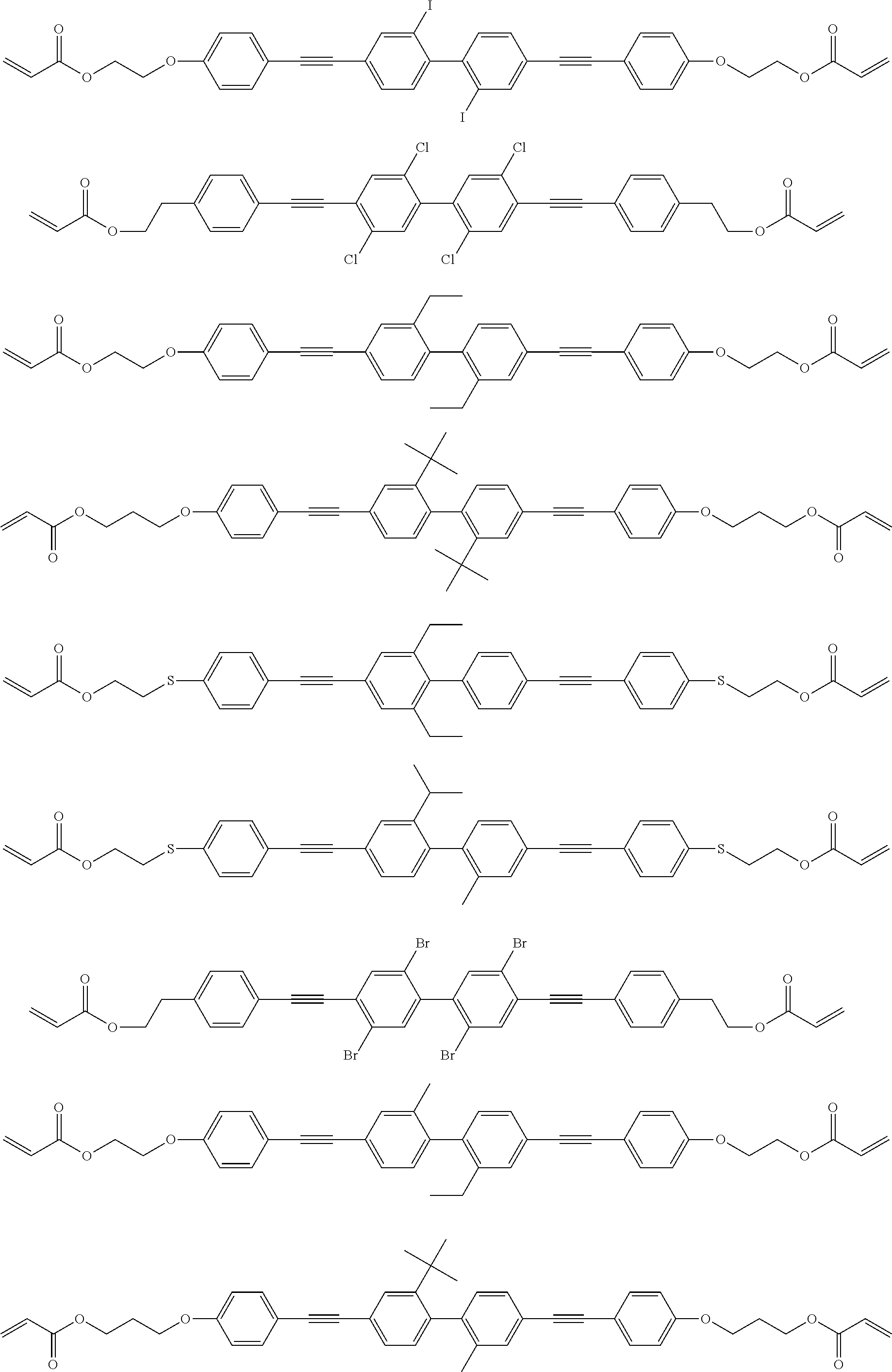

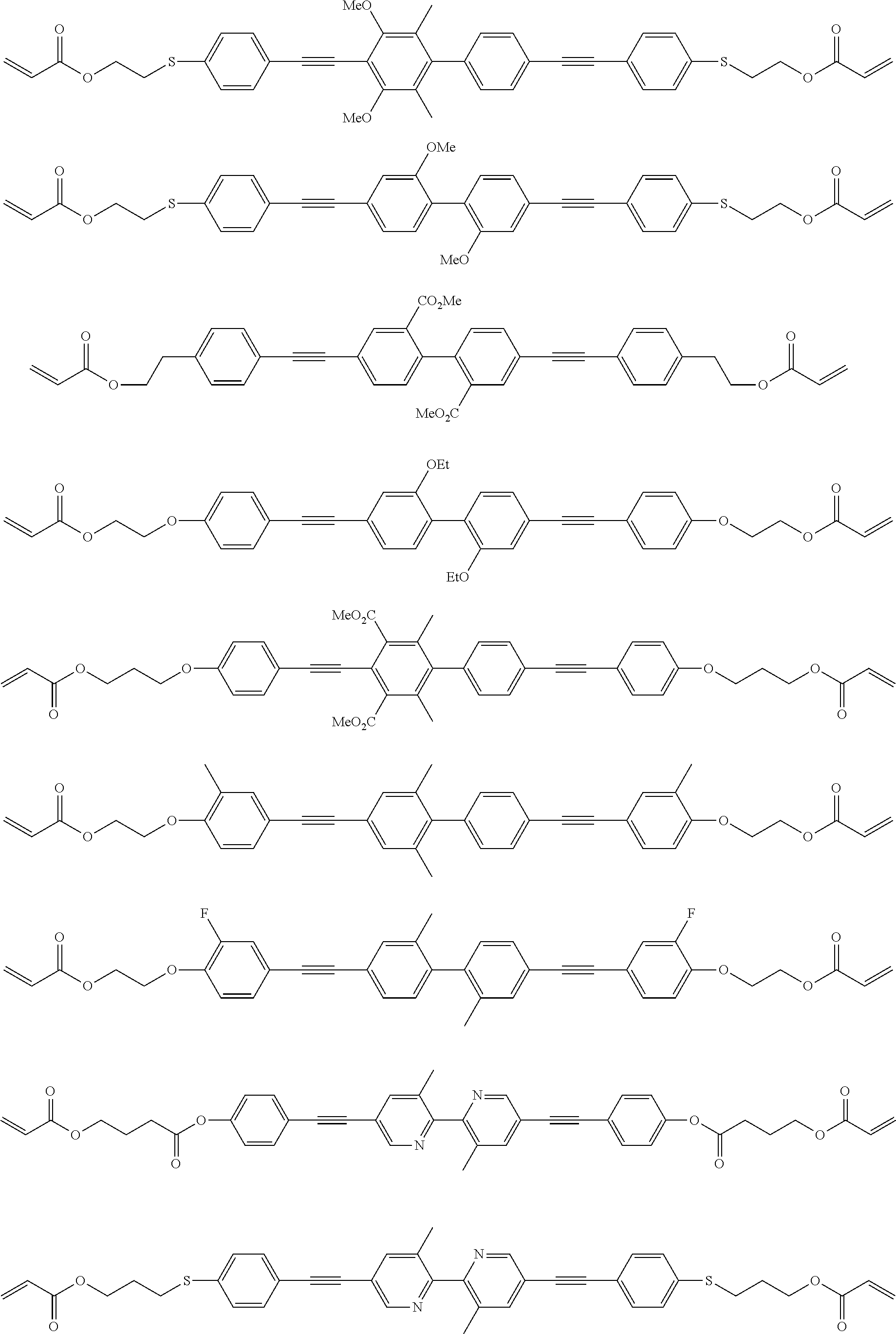

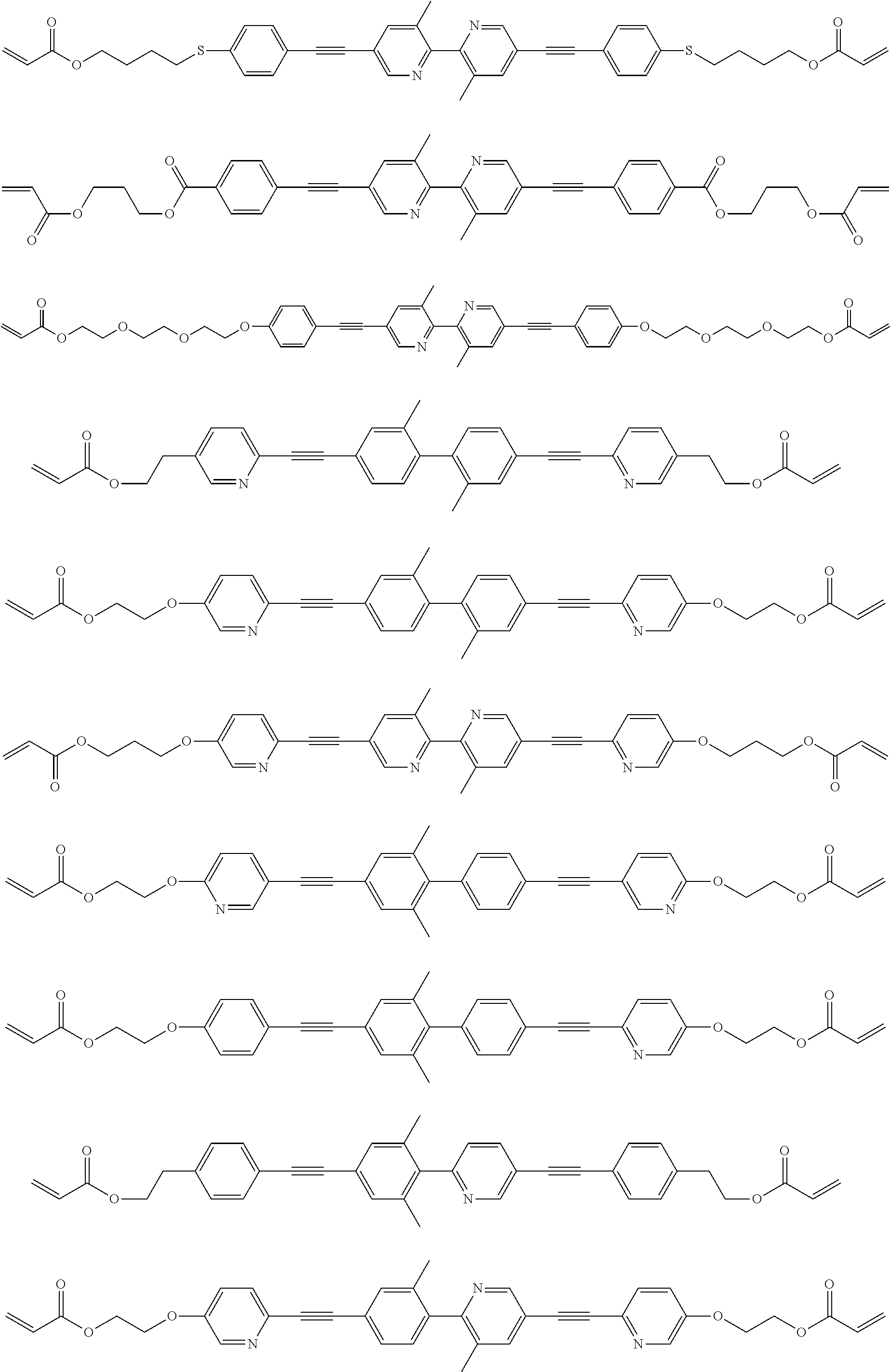

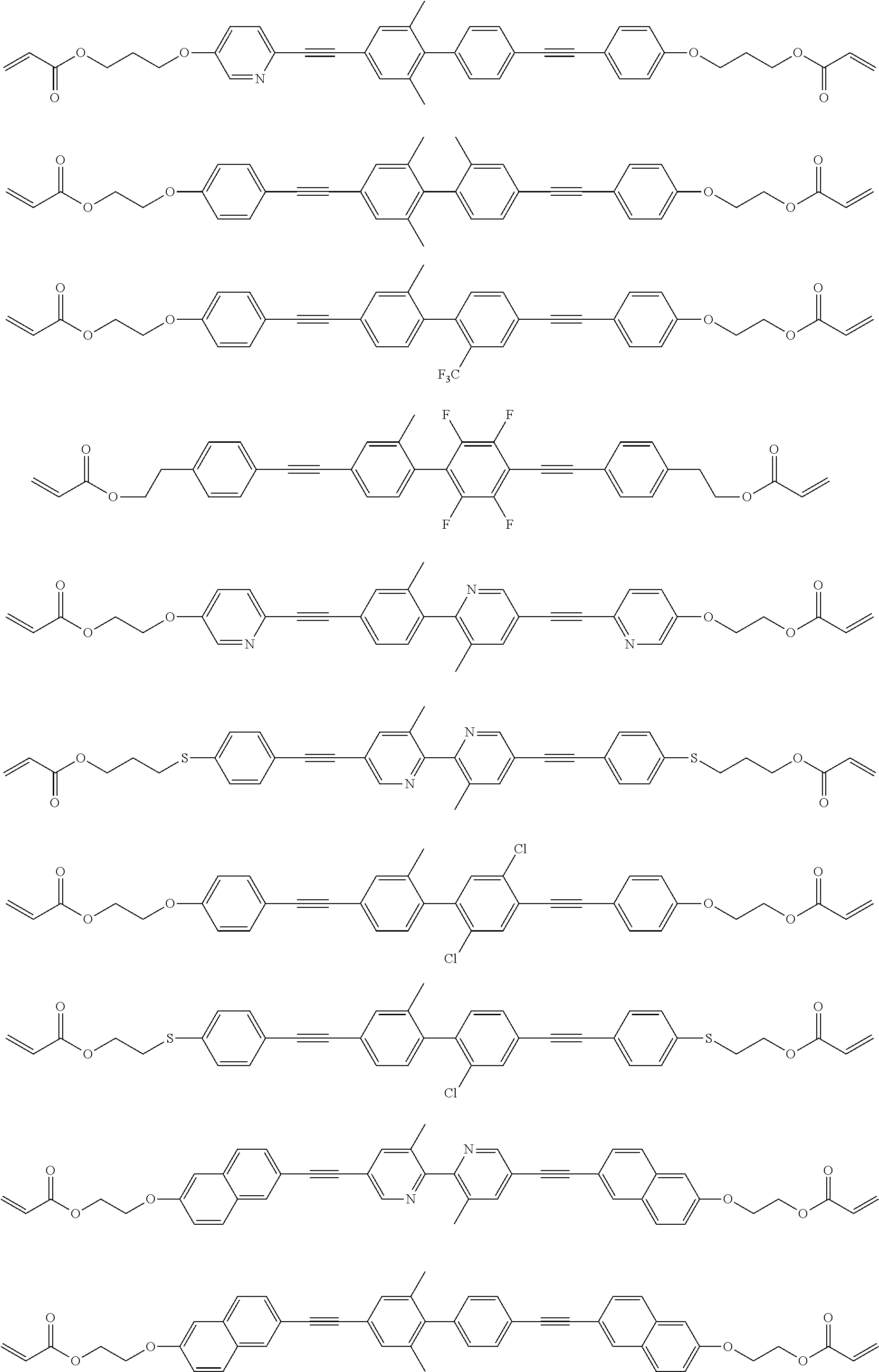

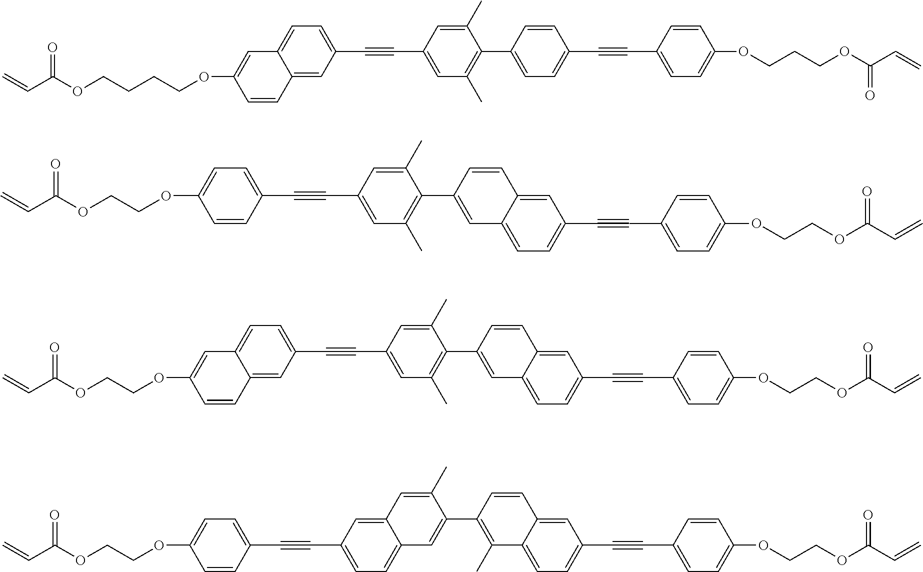

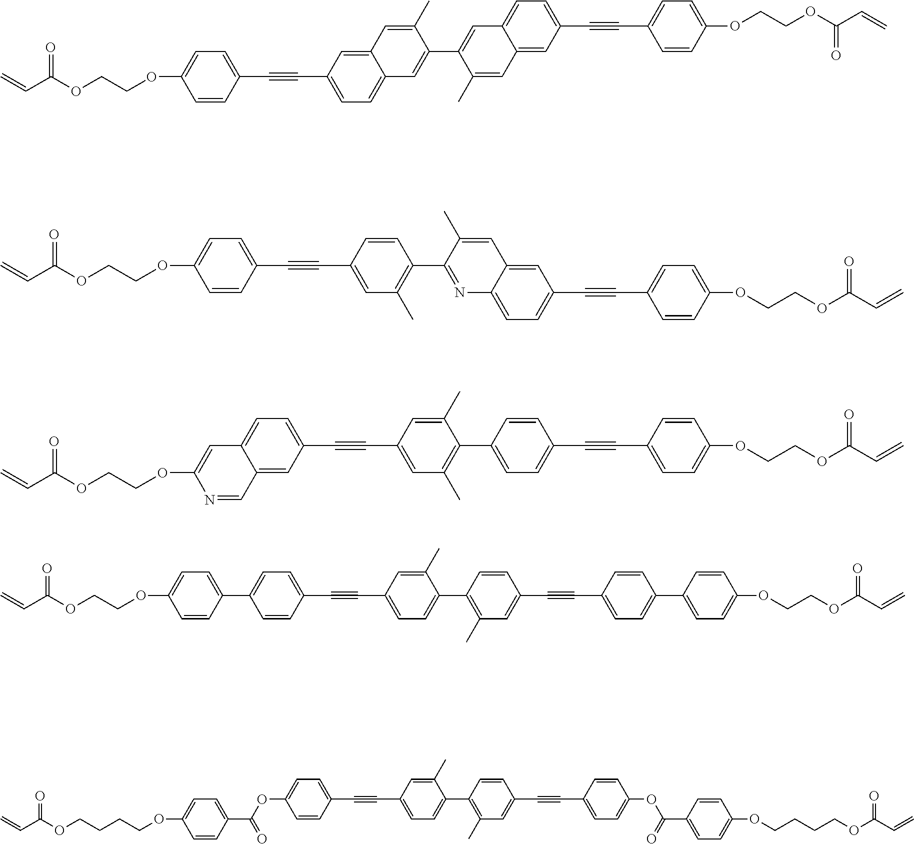

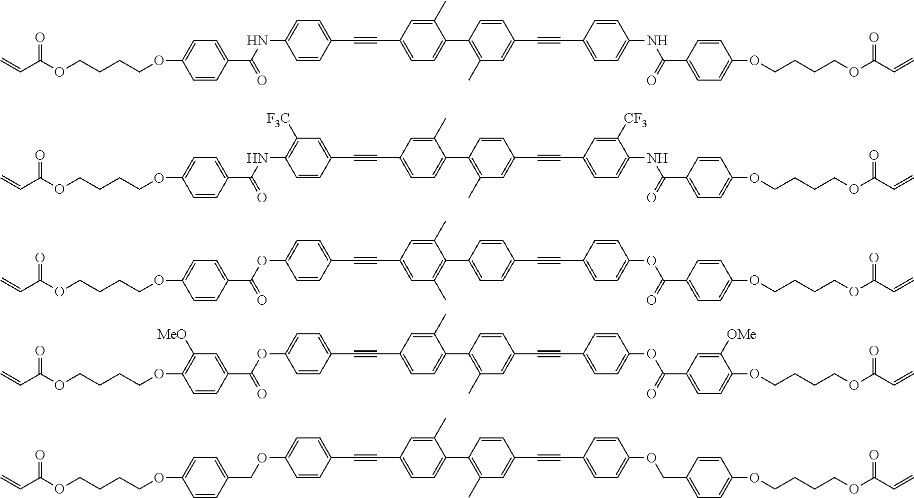

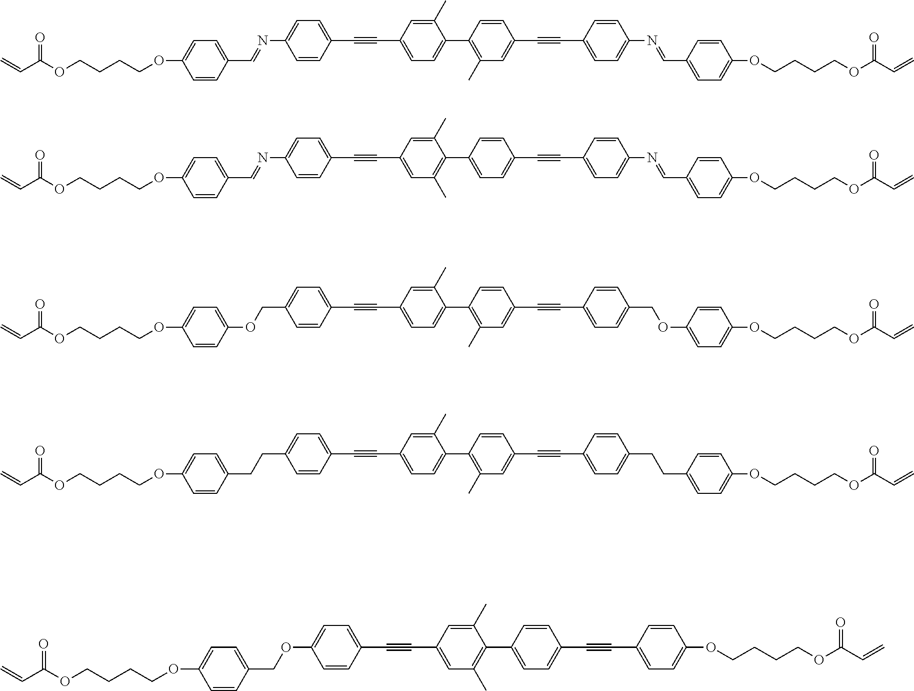

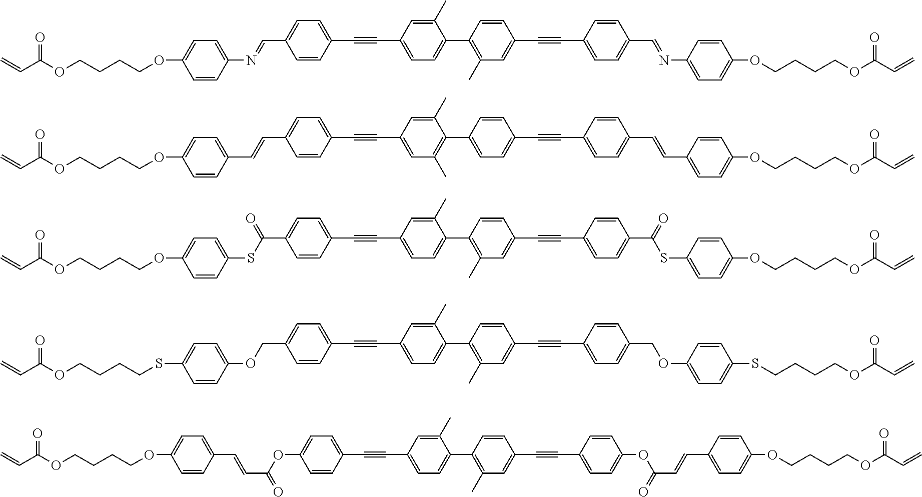

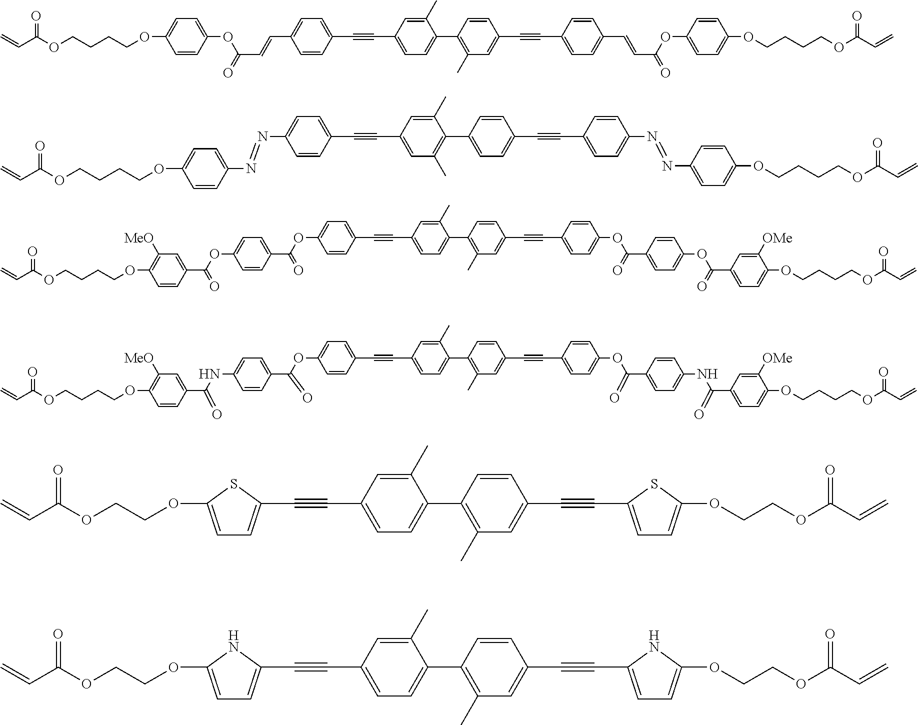

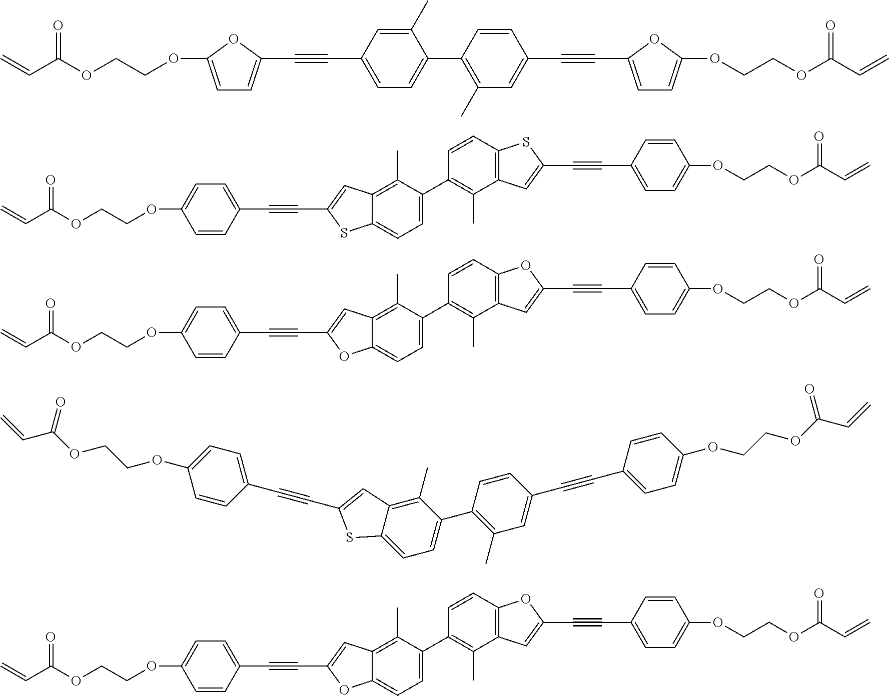

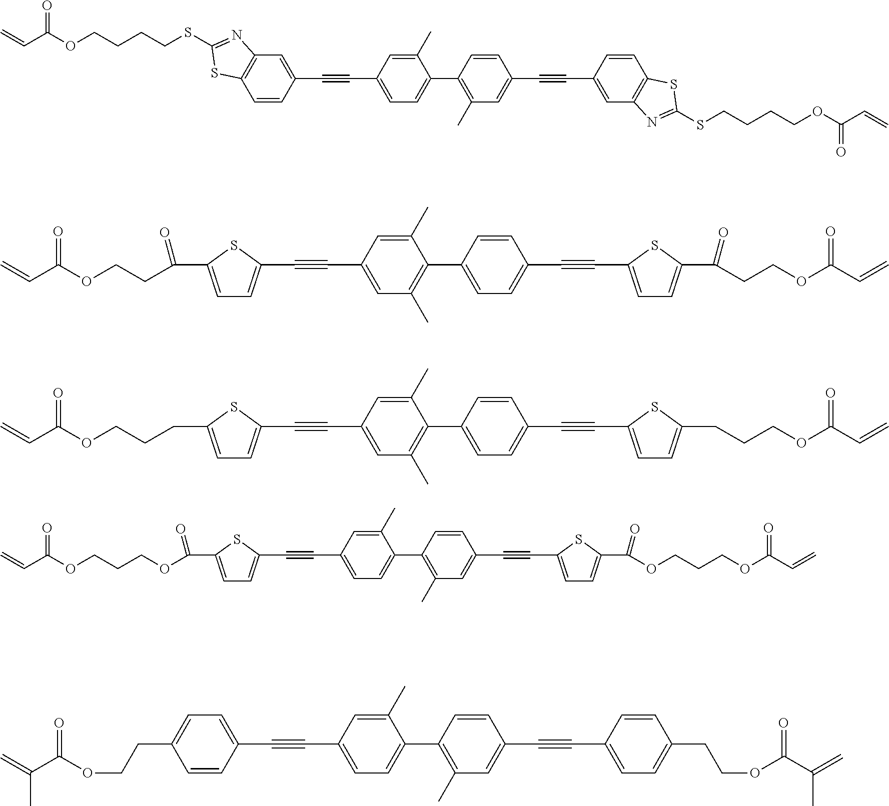

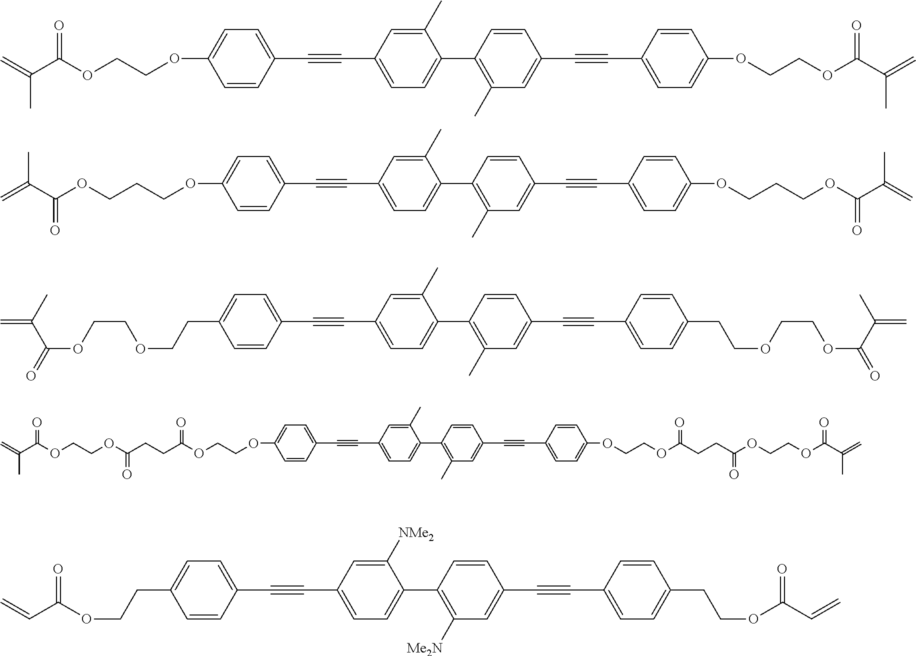

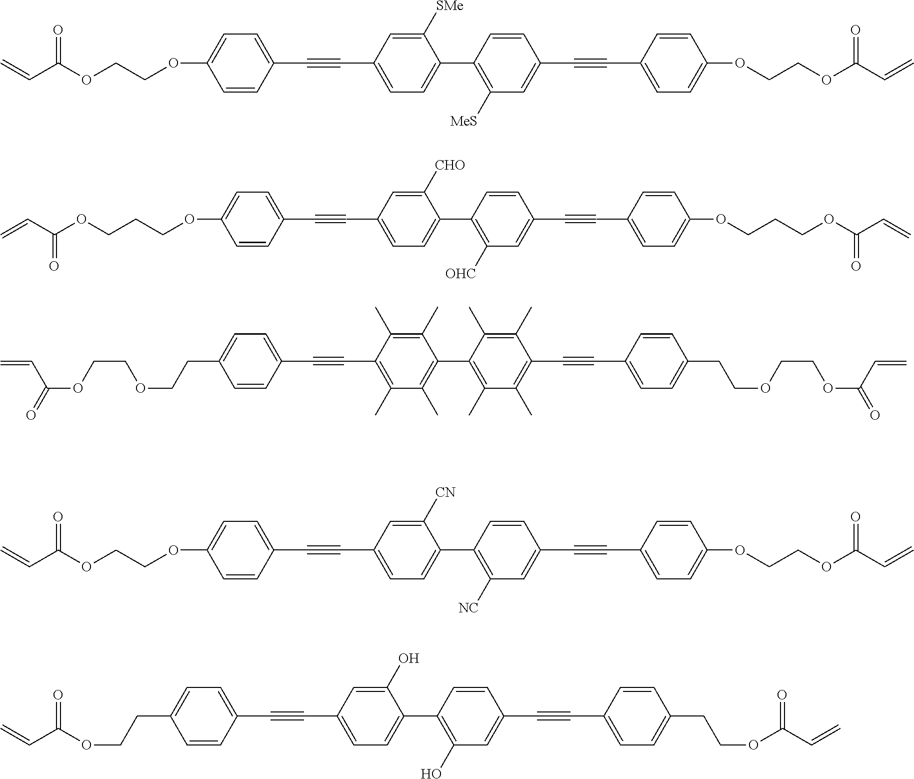

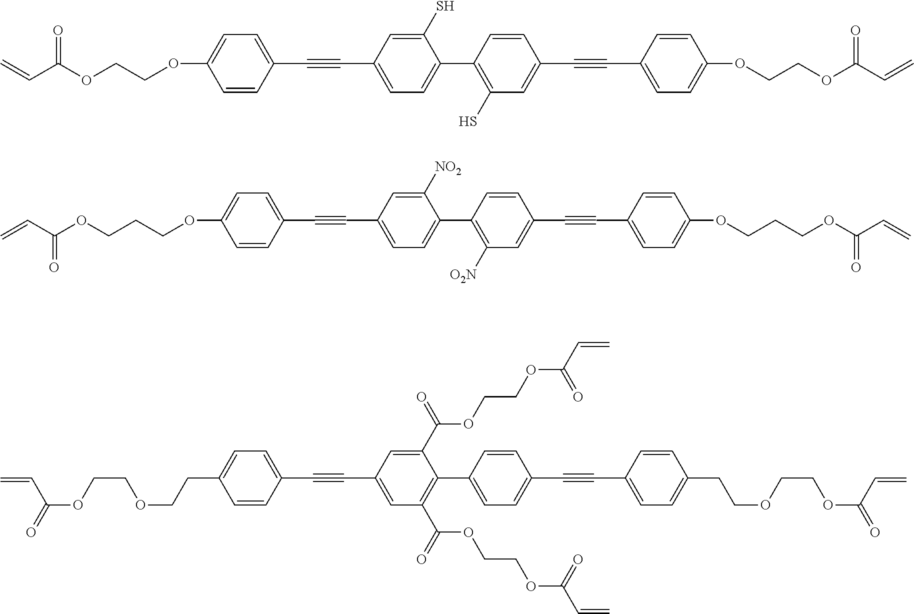

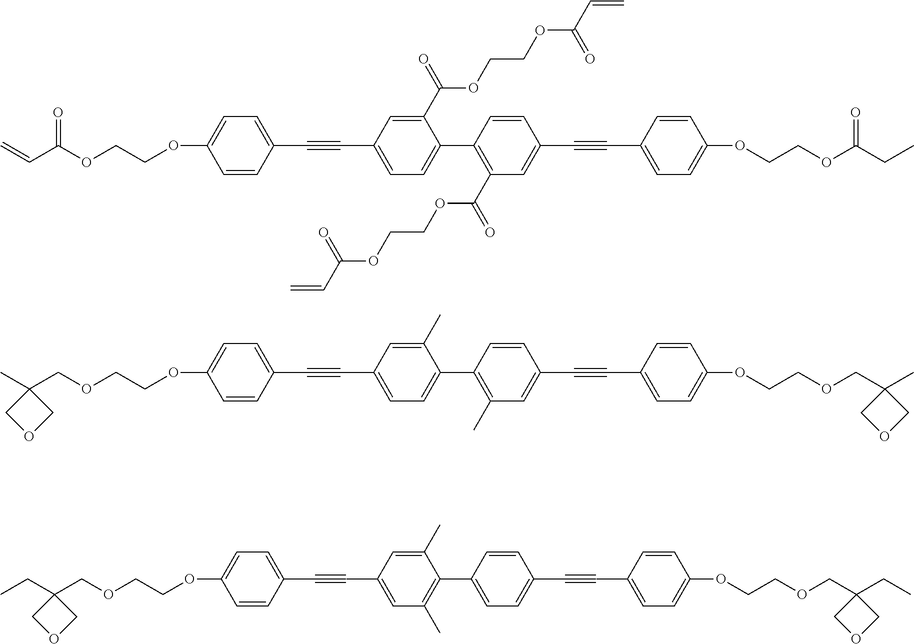

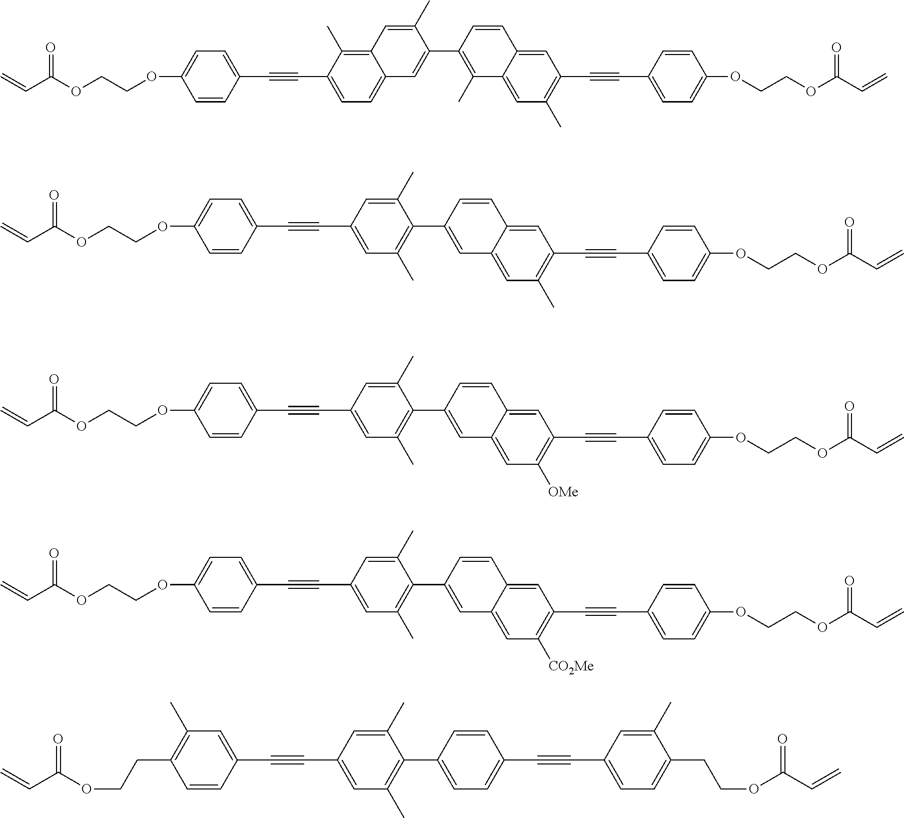

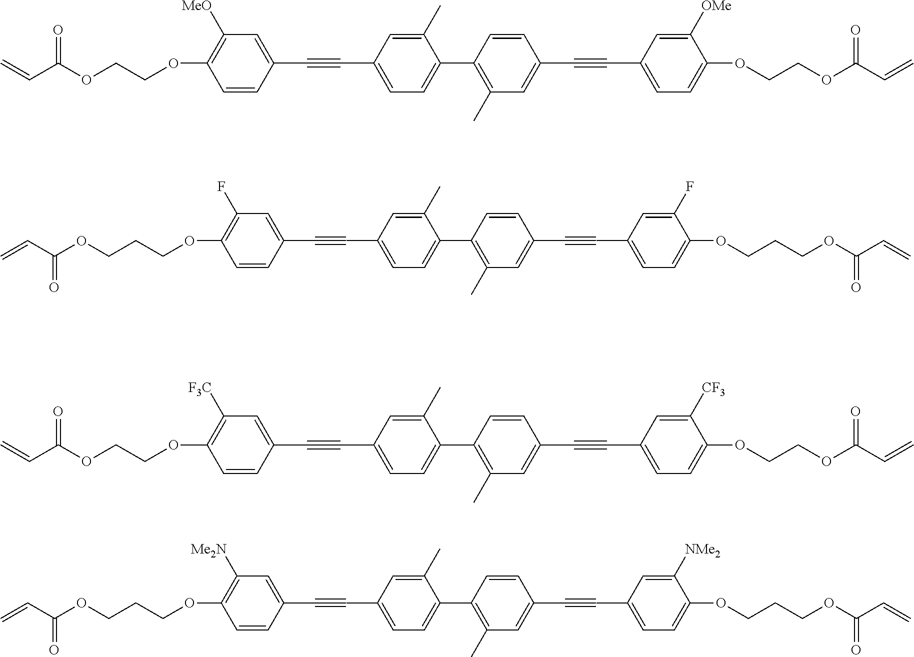

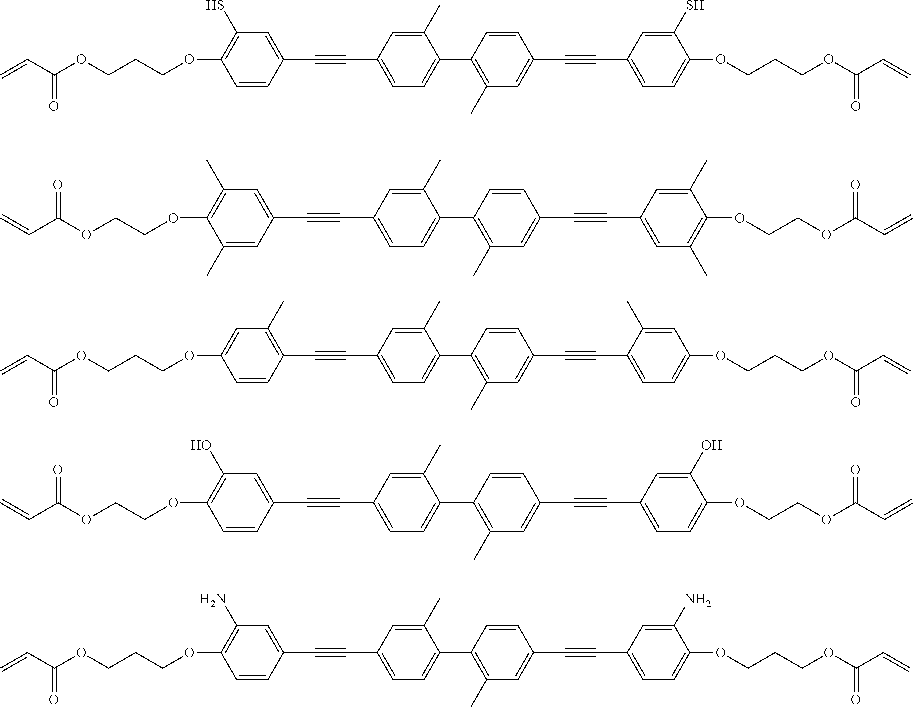

The compound represented by General Formula (I) can be synthesized with reference to a known method. Specific synthesis examples of the compound represented by General Formula (I) will be described in Examples described later.

The compound represented by General Formula (I) may have or may not have liquid crystallinity; however, it preferably has liquid crystallinity.

In a case where the compound represented by General Formula (I) has liquid crystallinity, it is easy to align the compound represented by General Formula (I) and it is possible to easily create a desired alignment pattern, which is preferable, in a case where an optically anisotropic layer is produced from a composition containing the compound represented by General Formula (I).

However, for example, in a case of being mixed with another compound having liquid crystallinity, the compound represented by General Formula (I) can be used to form a liquid crystal composition even in a case where the compound itself does not have liquid crystallinity, whereby a desired alignment pattern can be created.

That the compound has liquid crystallinity is intended to be that the compound has properties of exhibiting a mesophase between a crystal phase (a low temperature side) and an isotropic phase (a high temperature side) in a case where the temperature is changed. As a specific observation method, optical anisotropy and fluidity derived from the liquid crystal phase can be checked by carrying out an observation under a polarization microscope while heating or lowering the temperature of the compound with a hot stage or the like.

An optical element according to the embodiment of the present invention, which will be described later, is preferably produced by dissolving a composition containing the compound represented by General Formula (I) in a solvent and applying the dissolved composition. The precipitation concentration of the compound represented by General Formula (I) at 25° C. in a solvent is preferably 3% by mass or more and more preferably 10% by mass or more.

### Composition Containing Compound Represented by General Formula (I)

A composition containing the compound represented by General Formula (I) (hereinafter, also referred to as a “composition according to the embodiment of the present invention”) will be described.

The content of the compound represented by General Formula (I) in the composition according to the embodiment of the present invention is not particularly limited; however, it is preferably 5% to 100% by mass, more preferably 20% to 99% by mass, still more preferably 30% to 99% by mass, and particularly preferably 40% to 99% by mass, with respect to the total mass of the solid contents in the composition.

It is noted that the solid contents is intended to be components (non-volatile contents) other than a solvent in the composition. In a case of components other than the solvent, the components are regarded as the solid contents even in a case where they are components having properties of a liquid.

In the composition, one kind of the compound represented by General Formula (I) may be used alone, or two or more kinds thereof may be used. In a case where two or more kinds are used, the total content thereof is preferably within the above-described range.

The composition according to the embodiment of the present invention may have or may not have liquid crystallinity; however, it preferably has liquid crystallinity.

In a case where the composition according to the embodiment of the present invention has liquid crystallinity, it is easy to align the compound in the composition, and it is possible to easily produce a desired alignment pattern, which is preferable, in a case where an optically anisotropic layer is produced from the composition.

That the composition has liquid crystallinity is intended to be that the composition has properties of exhibiting a mesophase between a crystal phase (a low temperature side) and an isotropic phase (a high temperature side) in a case where the temperature is changed. As a specific observation method, optical anisotropy and fluidity derived from the liquid crystal phase can be checked by carrying out an observation under a polarization microscope while heating or lowering the temperature of the composition with a hot stage or the like.

The composition according to the embodiment of the present invention is preferably a composition for forming an optically anisotropic layer.

The composition according to the embodiment of the present invention may contain other components in addition to the compound represented by General Formula (I).

Hereinafter, the other components will be described.

### Another Liquid Crystal Compound

The composition according to the embodiment of the present invention may contain a liquid crystal compound (also referred to as “another liquid crystal compound”) that is not a compound represented by General Formula (I).

The other liquid crystal compound may be a rod-like liquid crystal compound or a disk-like liquid crystal compound; however, it is preferably a rod-like liquid crystal compound. In addition, the other liquid crystal compound is preferably a liquid crystal compound having a polymerizable group (other polymerizable liquid crystal compound).

Examples of the rod-like liquid crystal compound as the other liquid crystal compound include a rod-like nematic liquid crystal compound. The rod-like nematic liquid crystal compounds are preferably azomethines, azoxys, cyanobiphenyls, cyanophenyl esters, benzoic acid esters, cyclohexanecarboxylic acid phenyl esters, cyanophenylcyclohexanes, cyano-substituted phenylpyrimidines, alkoxy-substituted phenylpyrimidines, phenyldioxanes, tolans, or alkenylcyclohexylbenzonitriles. As the other liquid crystal compound, not only a liquid crystal compound having a low molecular weight but also a polymer liquid crystal compound can be used.

The liquid crystal compound having a polymerizable group can be obtained by introducing the polymerizable group into the liquid crystal compound. Examples of the polymerizable group include the polymerizable group exemplified as P¹ and P² of General Formula (I).

The liquid crystal compound having polymerizable groups preferably has 1 to 6 polymerizable groups and more preferably 1 to 3 polymerizable groups.

It is preferable that the other liquid crystal compound has a high refractive index anisotropy Δn, and specifically, the refractive index anisotropy Δn is preferably 0.15 or more, more preferably 0.18 or more, and still more preferably 0.22 or more. The upper limit thereof is not particularly limited; however, it is 0.60 or less in many cases.

In addition, by mixing the compound represented by General Formula (I) with the other liquid crystal compound and using the resultant mixture, the crystallization temperature as a whole can be significantly lowered.

Examples of other liquid crystal compounds include compounds disclosed in Makromol. Chem., Vol. 190, page 2255 (1989), Advanced Materials, Vol. 5, page 107 (1993), U.S. Pat. Nos. 4,683,327A, 4,983,479A, 5,622,648A, 5,770,107A, WO95/22586A, WO95/24455A, WO97/00600A, WO98/23580A, WO98/52905A, JP1989-272551A (JP-H1-272551A), JP1994-16616A (JP-H6-16616A), JP1995-110469A (JP-H7-110469A), JP1999-80081A (JP-H11-80081A), JP2001-328973A.

In a case where the composition according to the embodiment of the present invention contains another liquid crystal compound, the content of the other liquid crystal compound in the composition is not particularly limited; however, it is preferably 95% by mass or less, more preferably 1% to 80% by mass, still more preferably 1% to 70% by mass, and particularly preferably 1% to 60% by mass, with respect to the total mass of the solid contents in the composition.

In the composition according to the embodiment of the present invention, one kind of another liquid crystal compound may be used alone, or two or more kinds thereof may be used. In a case where two or more kinds are used, the total content thereof is preferably within the above-described range.

### Polymerization Initiator

The composition according to the embodiment of the present invention may contain a polymerization initiator.

The polymerization initiator is preferably a photopolymerization initiator which is capable of initiating a polymerization reaction by ultraviolet irradiation. Examples of the photopolymerization initiator include an α-carbonyl compound, an acyloin ether, an α-hydrocarbon-substituted aromatic acyloin compound, a polynuclear quinone compound, a phenazine compound, and an oxadiazole compound. In addition, a compound having an oxime ester structure is also preferable.

In a case where the composition according to the embodiment of the present invention contains a polymerization initiator, the content of the polymerization initiator in the composition is not particularly limited; however, it is preferably 0.1% to 20% by mass, and it is more preferably 1% to 8% by mass, with respect to the total mass of the compound represented by General Formula (I) (with respect to the total mass of the compound represented by General Formula (I) and another liquid crystal compound in a case where the composition contains the other liquid crystal compound).

In the composition according to the embodiment of the present invention, one kind of polymerization initiator may be used alone, or two or more kinds thereof may be used. In a case where two or more kinds are used, the total content thereof is preferably within the above-described range.

### Surfactant

The composition according to the embodiment of the present invention may contain a surfactant that contributes to the formation of a stable or rapid liquid crystal phase (for example, a nematic phase, a cholesteric phase).

Examples of the surfactant include a fluorine-containing (meth)acrylate-based polymer, compounds represented by General Formulae (X1) to (X3) disclosed in WO2011/162291A, compounds represented by General Formula (I) disclosed in paragraphs 0082 to 0090 of JP2014-119605A, and compounds disclosed in paragraphs 0020 to 0031 of JP2013-47204A.

Examples of the fluorine-containing (meth)acrylate-based polymer that can be used as a surfactant also include polymers disclosed in paragraphs 0018 to 0043 of JP2007-272185A.

In a case where the composition according to the embodiment of the present invention contains a surfactant, the content of the surfactant is not particularly limited; however, it is preferably 0.001% to 10% by mass and more preferably 0.05% to 3% by mass with respect to the total mass of the compound represented by General Formula (I) (with respect to the total mass of the compound represented by General Formula (I) and another liquid crystal compound in a case where the composition contains the other liquid crystal compound).

In the composition according to the embodiment of the present invention, one kind of surfactant may be used alone, or two or more kinds thereof may be used. In a case where two or more kinds are used, the total content thereof is preferably within the above-described range.

### Chiral Agent

The composition according to the embodiment of the present invention may contain a chiral agent. In a case where the composition according to the embodiment of the present invention contains a chiral agent, a cholesteric phase can be formed.

The kind of the chiral agent is not particularly limited. The chiral agent may be liquid crystalline or non-liquid crystalline. The chiral agent generally contains a chiral carbon atom. However, an axially chiral compound or a planar chiral compound, which does not contain any asymmetric carbon atom, can also be used as the chiral agent. Examples of the axially chiral compound or the planar chiral compound include binaphthyl, helicene, paracyclophane, and a derivative thereof. The chiral agent may have a polymerizable group.

In a case where the composition according to the embodiment of the present invention contains a chiral agent, the content of the chiral agent in the composition is not particularly limited; however, it is preferably 0.1% to 15% by mass and more preferably 1.0% to 10% by mass with respect to the total mass of the compound represented by General Formula (I) (with respect to the total mass of the compound represented by General Formula (I) and another liquid crystal compound in a case where the composition contains the other liquid crystal compound).

In the composition according to the embodiment of the present invention, one kind of chiral agent may be used alone, or two or more kinds thereof may be used. In a case where two or more kinds are used, the total content thereof is preferably within the above-described range.

### Solvent

The composition of the embodiment of the present invention may contain a solvent. The solvent is preferably a solvent capable of dissolving each component of the composition according to the embodiment of the present invention, and examples thereof include chloroform and methyl ethyl ketone. In a case where the composition according to the embodiment of the present invention contains the solvent, the content of the solvent in the composition is such an amount that the concentration of solid contents of the composition is preferably 0.5% to 20% by mass and more preferably 1% to 10% by mass.

In the composition according to the embodiment of the present invention, one kind of solvent may be used alone, or two or more kinds thereof may be used. In a case where two or more kinds are used, the total content thereof is preferably within the above-described range.

In addition to the above, the composition according to the embodiment of the present invention may also contain other components such as an antioxidant, an ultraviolet absorber, a sensitizer, a stabilizer, a plasticizer, a chain transfer agent, a polymerization inhibitor, an anti-foaming agent, a leveling agent, a thickener, a flame retardant, a surfactant, a dispersing agent, and a coloring material such as a dye or a pigment.

In addition, it is also preferable that the optically anisotropic layer is made to be responsive to substantially a wide range of wavelengths of incident light by imparting a twisting component to the composition according to the embodiment of the present invention or by laminating different retardation layers. For example, JP2014-089476A or the like discloses a method of realizing a λ/2 plate having a wide-range pattern by laminating two liquid crystal layers having different twisted directions in an optically anisotropic layer, which can be preferably used in the optical element according to the embodiment of the present disclosure.

### Cured Product and Optically Anisotropic Body

A cured product that is obtained by curing the composition according to the embodiment of the present invention, and an optically anisotropic body will be described.

A method of curing (polymerizing and curing) the composition according to the embodiment of the present invention is not particularly limited, and a known method can be adopted. Examples thereof include an aspect having a step X of bringing a predetermined substrate into contact with a composition to form a composition layer on the substrate and a step Y of subjecting the composition layer to a heat treatment so that the compound represented by General Formula (I) is aligned and then carrying out a curing treatment. According to the present aspect, the compound represented by General Formula (I) can be immobilized in an aligned state, whereby an optically anisotropic body (for example, an optically anisotropic layer) can be formed.

Hereinafter, procedures for the step X and the step Y will be described in detail.

The step X is a step of bringing a predetermined substrate into contact with a composition to form a composition layer on the substrate. The kind of the substrate to be used is not particularly limited, and examples thereof include known substrates (for example, a resin substrate, a glass substrate, a ceramic substrate, a semiconductor substrate, and a metal substrate).

The method of bringing a substrate into contact with a composition is not particularly limited, and examples thereof include a method of applying a composition onto a substrate and a method of immersing a substrate in a composition.

It is noted that after bringing a substrate into contact with a composition, as necessary, a drying treatment may be carried out in order to remove a solvent from the composition layer on the substrate.

The step Y is a step of subjecting the composition layer to a heat treatment so that the compound represented by General Formula (I) invention is aligned, and then carrying out a curing treatment.

In a case where the composition layer is subjected to a heat treatment, the compound represented by General Formula (I) is aligned and a liquid crystal phase is formed. For example, in a case where the composition layer contains a chiral agent, a cholesteric liquid crystalline phase is formed.

The conditions for the heat treatment are not particularly limited, and optimal conditions are selected depending on the kind of the compound represented by General Formula (I).

The method for the curing treatment is not particularly limited, and examples thereof include a photo-curing treatment and a thermal-curing treatment. Among these, a light irradiation treatment is preferable, and an ultraviolet irradiation treatment is more preferable.

For ultraviolet irradiation, a light source such as an ultraviolet lamp is used.

The cured product that is obtained by the above treatment corresponds to a layer that is obtained by immobilizing a liquid crystal phase. In particular, in a case where the composition contains a chiral agent, a layer is formed in which a cholesteric liquid crystalline phase is immobilized.

It is noted that these layers do not need to exhibit liquid crystallinity anymore. More specifically, for example, as a state in which the cholesteric liquid crystalline phase is “immobilized”, the most typical and preferred aspect is a state in which the alignment of the compound represented by General Formula (I) which is in the cholesteric liquid crystalline phase is retained. More specifically, it is preferably a state in which within a temperature range of usually 0° C. to 50° C., or −30° C. to 70° C. under the more severe conditions, no fluidity is exhibited in the layer, no changes in alignment form occur due to an external field or an external force, and a fixed alignment form can be kept stably and continuously.

### Optical Element

The optical element according to the embodiment of the present invention is an optical element that has an optically anisotropic layer formed from the above-described composition according to the embodiment of the present invention,
where the optically anisotropic layer has an alignment pattern, and
the alignment pattern is an alignment pattern in which an orientation of an optical axis, derived from a compound contained in the composition, continuously changes rotationally along at least one in-plane direction.

It is preferable that the alignment pattern is an alignment pattern in which the orientation of the optical axis, derived from the compound represented by General Formula (I), continuously changes rotationally along at least one in-plane direction, or an alignment pattern in which the orientation of the optical axis, derived from the compound represented by General Formula (I) and the other liquid crystal compound, continuously changes rotationally along at least one in-plane direction.

The optical element according to the embodiment of the present invention has an alignment pattern in which the orientation of the optical axis continuously changes rotationally along at least one in-plane direction, and thus it can diffract the light incident on the optical element. Since the compound represented by General Formula (I) is a compound having a high refractive index anisotropy Δn, the diffraction efficiency can be increased.

For the optical element, the description of paragraphs [0067] to [0107] of WO2020/022496A can be referred to.

The optical element according to the embodiment of the present invention can be applied as an optical member such as an augmented reality (AR) image projection device.

### Light Guide Element

A light guide element according to the embodiment of the present invention includes the above-described optical element and a light guide plate.

### EXAMPLES

Hereinafter, the present invention will be described more specifically with reference to Examples and Comparative Examples. In Examples below, the material, the using amount, the proportion, the details of treatment, the treatment procedure, and the like can be suitably modified without departing from the scope of the present invention. Accordingly, the scope of the present invention should not be interpreted restrictively by the following specific examples.

Synthesis examples of compounds A-1 to A-6 used in Examples are shown below.

### Synthesis of Compound

### Synthesis Example 1: Synthesis of Compound A-1

The compound A-1 was synthesized according to the following scheme. It is noted that a compound 1 was synthesized according to WO2019/182129A.

In addition, r.t. indicates room temperature.

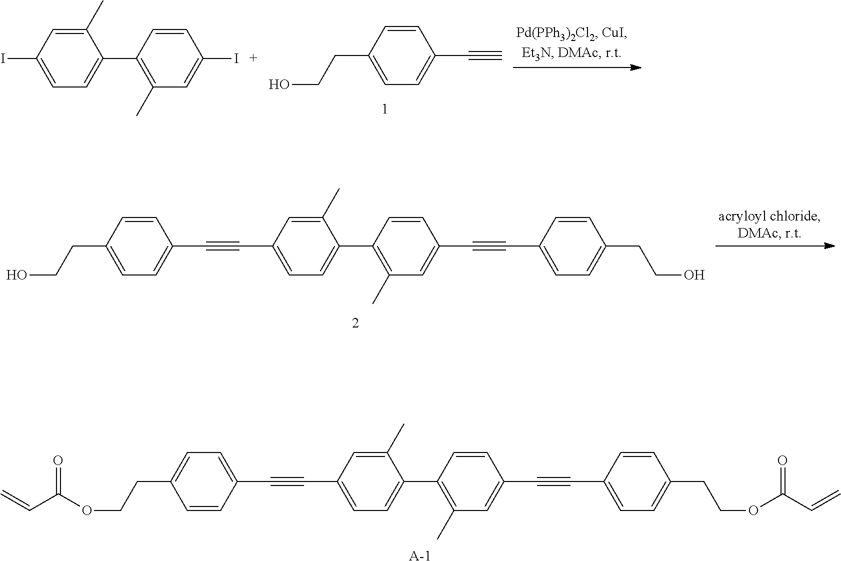

### (1) Synthesis of Compound 2

4,4′-diiodo-2,2-dimethylbiphenyl (3.00 g, 6.91 mmol) and the compound 1 (2.34 g, 16.0 mmol) were dissolved in dimethylacetamide (DMAc) (30 mL) in a nitrogen atmosphere, and triethylamine (6.99 g, 69.1 mmol) was added thereto. After nitrogen bubbling of the obtained solution for 1 hour, Pd(PPh₃)₂Cl₂ (243 mg, 0.346 mmol) and CuI (132 mg, 0.693 mmol) were added thereto, and the resultant mixture was stirred at room temperature (25° C.) for 1 hour. The obtained solution was cooled in an ice water bath, 1 N hydrochloric acid (60 mL) and MeOH (50 mL) were added thereto, the resultant mixture was stirred at the above temperature for 30 minutes, and the precipitate was filtered. The obtained solid was dissolved in ethyl acetate (100 mL) and tetrahydrofuran (THF) (40 mL) and sequentially washed with 1 N hydrochloric acid, a sodium bicarbonate solution, and then a saline solution. Then the obtained organic layer was dried with magnesium sulfate. After filtering the organic layer, the solvent was distilled off under reduced pressure, and the obtained residue was purified by flash column chromatography to obtain a compound 2 (2.78 g, 5.91 mmol). The yield was 85.5%.

### (2) Synthesis of Compound A-1

The compound 2 (2.00 g, 4.25 mmol) was dissolved in dimethylacetamide (DMAc) (50 mL). The obtained solution was cooled in an ice water bath, acryloyl chloride (1.54 g, 17.0 mmol) was added thereto, and stirring was carried out at room temperature for 3 hours. The obtained solution was cooled in an ice water bath, acryloyl chloride (0.34 g, 3.71 mmol) was added thereto again, and stirring was carried out at room temperature for 2 hours. The obtained solution was cooled in an ice water bath, ethyl acetate (200 mL) and water (200 mL) were added thereto, and then extraction was carried out with ethyl acetate. The obtained organic layer was sequentially washed with a sodium bicarbonate solution and then a saline solution, followed by being dried with magnesium sulfate. After filtering the organic layer, the solvent was distilled off under reduced pressure, and the obtained residue was purified by flash column chromatography, whereby the compound A-1(2.05 g, 3.54 mmol) was obtained. The yield was 83.3%.

¹H-NMR (CDCl₃): δ=2.06 (s, 6H), 3.01 (t, 4H), 4.39 (t, 4H), 5.83 (d, 2H), 6.12 (dd, 2H), 6.42 (d, 2H), 7.08 (d, 2H), 7.23 (d, 4H), 7.39 (d, 2H), 7.41-7.50 (m, 6H)

### Synthesis Example 2: Synthesis of Compound A-2

The compound A-2 was synthesized according to the following scheme. It is noted that a compound 3 was synthesized according to WO2011/050276A.

TBSO indicates a group in which a hydroxyl group is protected by a tert-butyldimethylsilyl group, and TMS indicates a trimethylsilyl group (—Si(CH₃)₃).

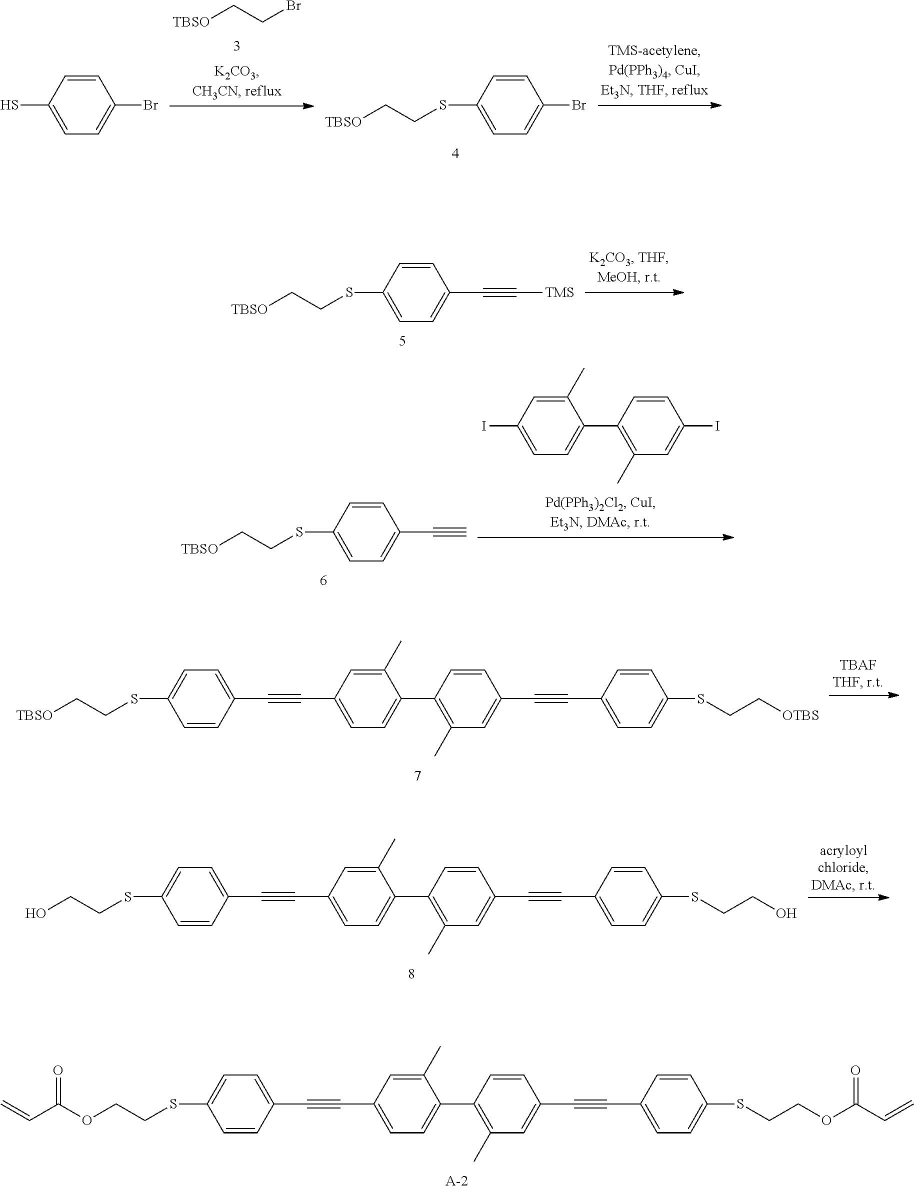

### (1) Synthesis of Compound 4

4-bromothiophenol (28.0 g, 0.148 mol) and the compound 3 (36.5 g, 0.148 mmol) were dissolved in acetonitrile (500 mL), potassium carbonate (40.9 g, 0.296 mol) was added thereto, and the resultant mixture was stirred with heating under reflux for 2 hours. The obtained solution was cooled in an ice water bath, ethyl acetate (500 mL) and water (400 mL) were added thereto, and then extraction was carried out with ethyl acetate. The obtained organic layer was dried with magnesium sulfate, and the organic layer was filtered. The solvent was distilled off under reduced pressure, and the obtained residue was purified by flash column chromatography, whereby a compound 4 (52.2 g, 0.150 mol) was obtained. The yield was 62.4%.

### (2) Synthesis of Compound 5

The compound 4 (32.0 g, 92.1 mmol) was dissolved in THF (320 mL), and triethylamine (92.8 g, 0.917 mol) was added thereto. After nitrogen bubbling of the obtained solution for 1 hour, trimethylsilyl acetylene (TMS-acetylene) (10.9 g, 0.110 mol), Pd(PPh₃)₄ (2.12 g, 1.83 mmol) and CuI (0.35 g, 1.8 mmol) were added thereto, and the resultant mixture was stirred with heating under reflux for 4 hours. The obtained solution was filtered and sequentially washed with water, 1 N hydrochloric acid, a sodium bicarbonate solution, and then a saline solution. The obtained organic layer was dried with magnesium sulfate, and the organic layer was filtered. The solvent was distilled off under reduced pressure, and the obtained residue was purified by flash column chromatography, whereby a compound 5 (28.4 g, 77.9 mmol) was obtained. The yield was 84.9%.

### (3) Synthesis of Compound 6

The compound 5 (28.4 g, 77.9 mmol) was dissolved in a mixed solution of THF (140 mL) and MeOH (140 mL), potassium carbonate (31.7 g, 0.229 mol) was added thereto, and the resultant mixture was stirred at room temperature for 1 hour. Water was added to the obtained solution, and then extraction was carried out with ethyl acetate. The obtained organic layer was washed with a saline solution and then dried with magnesium sulfate. After filtering the organic layer, the solvent was distilled off under reduced pressure, and the obtained residue was purified by flash column chromatography, whereby a compound 6 (20.5 g, 70.1 mmol) was obtained. The yield was 91.8%.

### (4) Synthesis of Compound 7

4,4′-diiodo-2,2-dimethylbiphenyl (1.50 g, 3.46 mmol) and the compound 6 (2.22 g, 7.60 mmol) were dissolved in DMAc (15 mL) in a nitrogen atmosphere, and triethylamine (3.55 g, 34.6 mmol) was added thereto. After nitrogen bubbling of the obtained solution for 1 hour, Pd(PPh₃)₂Cl₂ (121 mg, 0.172 mmol) and CuI (66 mg, 0.347 mmol) were added thereto, and the resultant mixture was stirred at room temperature for 1 hour. The obtained solution was cooled in an ice water bath, 1 N hydrochloric acid (60 mL) and MeOH (50 mL) were added thereto, and then extraction was carried out with ethyl acetate. The obtained organic layer was sequentially washed with 1 N hydrochloric acid, a sodium bicarbonate solution, and then a saline solution, followed by being dried with magnesium sulfate. After filtering the organic layer, the solvent was distilled off under reduced pressure, and the obtained residue was purified by flash column chromatography, whereby a compound 7 (2.24 g, 2.93 mmol) was obtained. The yield was 84.8%.

### (5) Synthesis of Compound 8

The compound 7 (2.20 g, 2.88 mmol) was dissolved in THF (10 mL). The obtained solution was cooled in an ice water bath, a THF solution of tetra-n-butylammonium fluoride (TBAF) (1 mol/L, 6.1 mL, 6.1 mmol) was added thereto, and stirring was carried out at room temperature for 2 hours. The obtained solution was cooled in an ice water bath, ethyl acetate (50 mL) and water (50 mL) were added thereto, and then extraction was carried out with ethyl acetate. The obtained organic layer was sequentially washed with a saline solution and then dried with magnesium sulfate. After filtering the organic layer, the solvent was distilled off under reduced pressure. The obtained residue was dissolved in ethyl acetate, hexane was added thereto, and a reprecipitation treatment was carried out to obtain a compound 8 (1.48 g, 2.77 mmol). The yield was 96.1%.

### (6) Synthesis of Compound A-2

The compound 8 (1.40 g, 2.62 mmol) was dissolved in DMAc (20 mL). The obtained solution was cooled in an ice water bath, acryloyl chloride (1.18 g, 13.1 mmol) was added thereto, and stirring was carried out at room temperature for 2 hours, followed by stirring at 40° C. for 1 hour. The obtained solution was cooled in an ice water bath, ethyl acetate (100 mL) and water (100 mL) were added thereto, and then extraction was carried out with ethyl acetate. The obtained organic layer was sequentially washed with 1 N hydrochloric acid, a sodium bicarbonate solution, and then a saline solution, followed by being dried with magnesium sulfate. After filtering the organic layer, the solvent was distilled off under reduced pressure, and the obtained residue was purified by flash column chromatography, whereby the compound A-2 (1.40 g, 2.18 mmol) was obtained. The yield was 83.3%.

¹H-NMR (CDCl₃): δ=2.06 (s, 6H), 3.23 (t, 4H), 4.36 (t, 4H), 5.845 (d, 1H), 5.855 (d, 1H), 6.10 (dd, 2H), 6.40 (d, 2H), 7.09 (d, 2H), 7.35-7.39 (m, 6H), 7.41-7.48 (m, 6H)

### Synthesis Example 3: Synthesis of Compound A-3

A compound A-3 was obtained according to the same procedure as in Synthesis Example 1, except that 4-(4-ethynylphenyloxy)butan-1-ol synthesized according to WO2018/034216A was used instead of the compound 1.

### Synthesis Example 4: Synthesis of Compound A-4

A compound A-4 was obtained according to the same procedure as in Synthesis Example 1, except that 4-(4-ethynylphenyloxy)butan-1-ol synthesized according to WO2018/034216A was used instead of 4,4′-diiodo-2,2-dimethylbiphenyl.

### Synthesis Example 5: Synthesis of Compound A-5

A compound A-5 was obtained according to the same procedure as in Synthesis Example 1, except that 4,4′-diiodo-2,2′,6,6′-1,1′-biphenyl synthesized according to David, V. et al., Eur. J. Org. Chem. 2010, 120 (2010) was used instead of 4,4′-diiodo-2,2-dimethylbiphenyl.

### Synthesis Example 6: Synthesis of Compound A-6

A compound A-6 was obtained according to the same procedure as in Synthesis Example 1, except that 5,5′-dibromo-3,3′-dimethyl-2,2′-bipyridine synthesized according to Muraoka, T. et al., JACS, 139, 18016 (2017) was used instead of 4,4′-diiodo-2,2-dimethylbiphenyl.

### Comparative Example 1: Synthesis of Compound B-1

A compound B-1 was obtained as a comparative compound according to the same procedure as in Synthesis Example 1, except that 4,4′-diiodobiphenyl was used instead of 4,4′-diiodo-2,2-dimethylbiphenyl.

### Comparative Example 2: Synthesis of Compound B-2

According to JP2005-15406A, a compound B-2 was obtained as a comparative compound.

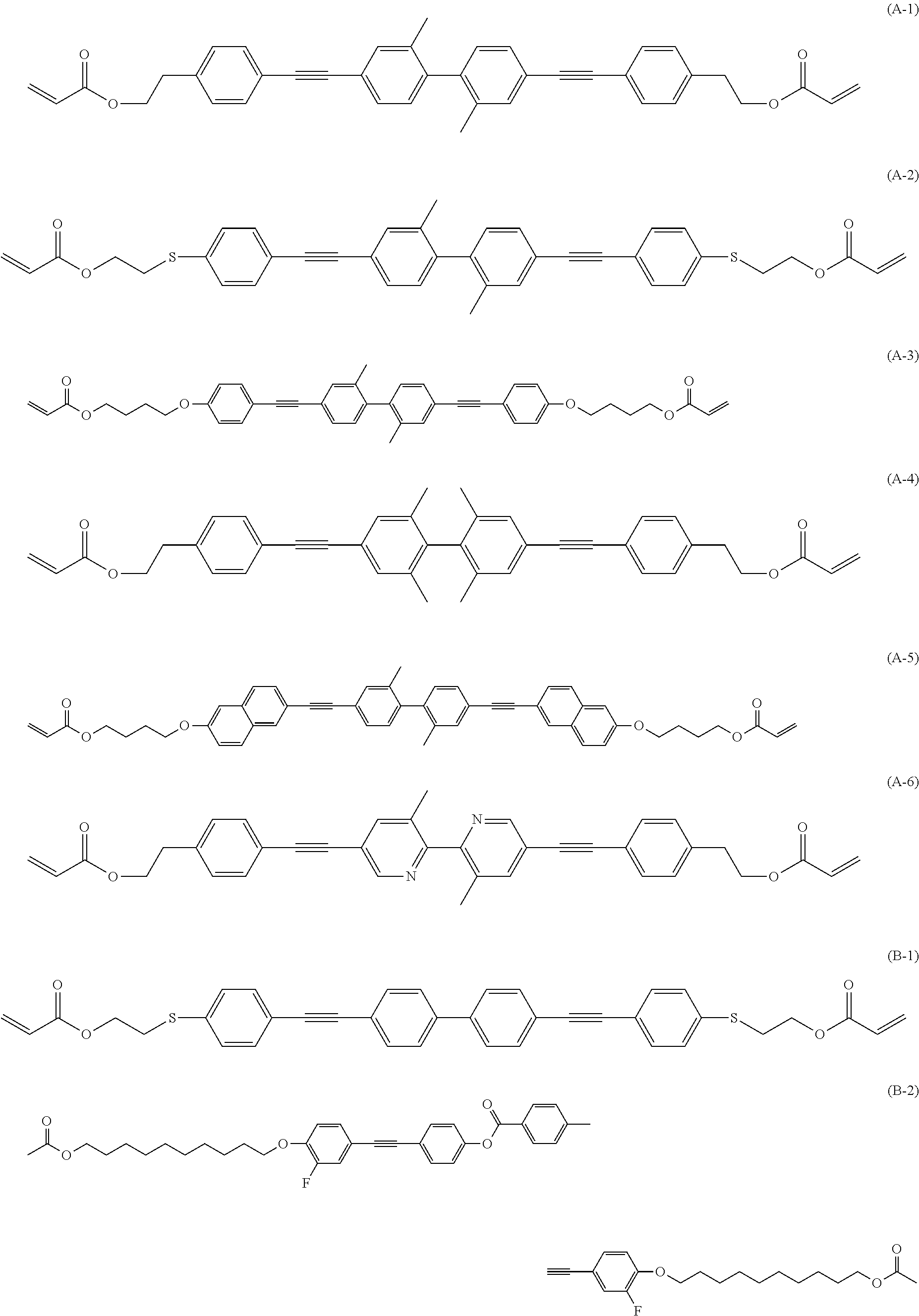

### Examples 1 to 6 and Comparative Examples 1 and 2

### [Evaluation]

Using the above-described compounds A-1 to A-6 and compounds B-1 and B-2, the following various evaluations were carried out.

The compounds shown in Table 1 were used in Examples 1 to 6 and Comparative Examples 1 and 2.

### <Evaluation of Liquid Crystallinity>

The respective compounds (the compounds A-1 to A-6 and the compounds B-1 and B-2) were heated on a hot stage and observed with a polarization microscope, and the phase transition temperature was measured to evaluate the presence or absence of the liquid crystallinity. A case of having liquid crystallinity was evaluated as A, and a case of having no liquid crystallinity was evaluated as B. The results are shown in Table 1.

Since B-1 has melting points of 200° C. or higher and undergo a polymerization reaction during heating, liquid crystallinity could not be evaluated.

### <Measurement of Δn (Refractive Index Anisotropy)>

The Δn of each of the compounds (the compounds A-1 to A-6 and the compounds B-1 and B-2), which are the compounds to be measured, was measured by a method using a wedge-shaped liquid crystal cell described on page 202 in the Liquid crystal handbook (edited by the liquid crystal handbook editorial board, published by Maruzen Co., Ltd., 2000). Δn was defined as a measured value at a wavelength of 550 nm at 30° C. or the lower limit temperature of the nematic phase+0° C. to 10° C. In a case of a compound which is liable to crystallize or a compound having no liquid crystallinity, evaluation was carried out using a mixture thereof with the other liquid crystal compound, and Δn was estimated from the extrapolated value of the measured value thereof.

The following L-1-1 was used as the other liquid crystal compound. As the above mixture, a mixture obtained by mixing at a ratio of the compound to be measured/the L-1-1=1/2 (in terms of mass ratio) was used.

A case where Δn was 0.35 or more was evaluated as A, a case where Δn was 0.30 or more and less than 0.35 was evaluated as B, and a case where Δn was less than 0.30 was evaluated as C. The results are shown in Table 1.

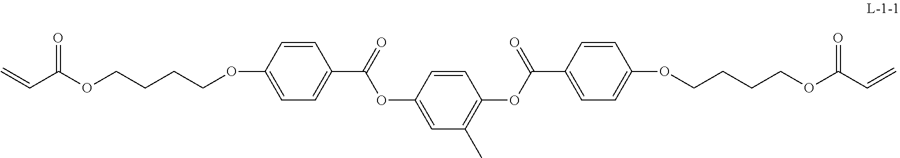 [SMILES: C=CC(=O)OCCCCOc1ccc(C(=O)Oc2ccc(OC(=O)c3ccc(OCCCCOC(=O)C=C)cc3)c(C)c2)cc1]

### <Evaluation of Solubility>

The solubility of each of the compounds (the compounds A-1 to A-6 and the compounds B-1 and B-2) in methyl ethyl ketone was evaluated. A solution in which the compound was ultrasonically dissolved or dissolved by heating was prepared, and then it was observed at room temperature (25° C.) whether or not the compound was precipitated in the solution. Solutions were prepared at various concentrations for each compound, the concentration at which the precipitation of the compound occurred was defined as the precipitation concentration, the solubility in a case where the precipitation concentration was 10% by mass or more was evaluated as A, the solubility in a case where the precipitation concentration was 3% by mass or more and less than 10% by mass was evaluated as B, and the solubility in a case where the precipitation concentration was less than 3% by mass was evaluated as C. The results are shown in Table 1.

### <Light Resistance Evaluation>

As shown below, the light resistance of the optically anisotropic layer produced using the composition containing each of the compounds A-1 to A-6 and the compounds B-1 and B-2 was evaluated.

### (Production of Optically Anisotropic Layer for Light Resistance Test)

A coating liquid having the following composition was prepared and applied by spin-coating, onto a rubbing-treated glass attached with an alignment film. Each composition was irradiated with ultraviolet rays of 300 mJ/cm² through a filter that cuts light having a wavelength of 350 nm or less on a hot plate which had been heated to a temperature at which a nematic phase is shown, whereby an optically anisotropic layer was produced.

Composition of coating liquid

| | | |
|---|---|---|
| A compound shown in Table 1 below | 25 | parts by mass |
| The following polymerizable liquid | 75 | parts by mass |
| crystal compound L-1 | | |
| A polymerization initiator | 2 | parts by mass |
| (IRGACURE (registered trade name) | | |
| 907, manufactured by BASF SE) | | |
| The following leveling agent T-1 | 0.1 | parts by mass |
| Chloroform | 1,940 | parts by mass |

The polymerizable liquid crystal compound L-1 is a mixture obtained by mixing at a ratio of the following L-1-1/L-1-2/L-1-3=84/14/2 (in terms of mass ratio).

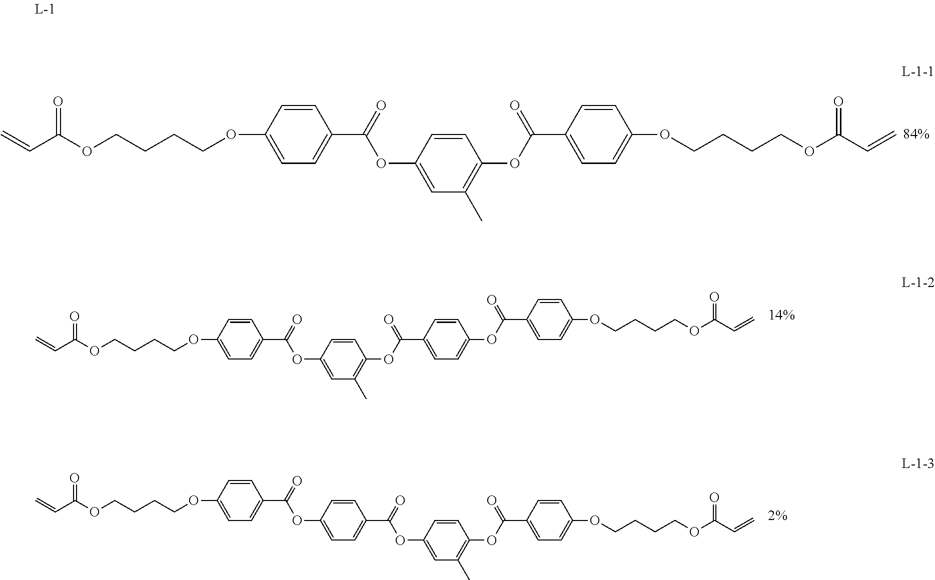

The leveling agent T-1 is a compound having the following structure.

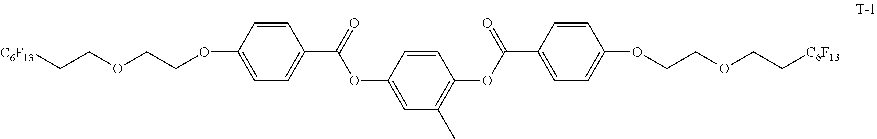

### (Evaluation of Light Resistance)

The produced optically anisotropic layers for a light resistance test were irradiated with light using a Super Xenon Weather Meter SX75 manufactured by Suga Test Instruments Co., Ltd. Using KU-1000100 manufactured by King Seisakusho Co., Ltd. as a UV cut filter, a light resistance test was carried out by irradiation with 5 million lx of light for 50 hours under an oxygen-blocking condition. The temperature of the specimen to be tested (the temperature inside the test device) was set to 63° C. The relative humidity in the test device was 50% RH.

The Re of the optically anisotropic layer before and after the light resistance test was measured, the light resistance in a case where the rate of change in Re described below was less than 10% was evaluated as A, and the light resistance in a case where the rate of change in Re was 10% or more was evaluated as B. The smaller the rate of change in Re, the more excellent the light resistance. The results are shown in Table 1.

It is noted that Re is the in-plane retardation.

Rate of change in Re (%)=[100×{|(Re after test)−(Re before test)|}/(Re before test)]

Re was measured with Axoscan manufactured by Axometrics Inc. at a wavelength of 550 nm, and the measurement temperature was set to room temperature.

TABLE 1

| | | Liquid | | | Light |
|---|---|---|---|---|---|
| | Compound | crystallinity | Δn | Solubility | resistance |
| Example 1 | A-1 | A | B | A | A |
| Example 2 | A-2 | A | A | A | A |
| Example 3 | A-3 | A | B | A | A |
| Example 4 | A-4 | A | B | A | A |
| Example 5 | A-5 | A | A | B | A |
| Example 6 | A-6 | A | B | A | A |
| Comparative | B-1 | — | A | C | B |
| Example 1 | | | | | |
| Comparative | B-2 | A | C | B | B |
| Example 2 | | | | | |

From the results shown in Table 1 above, it was found that the compound represented by General Formula (I) has a high refractive index anisotropy Δn and has excellent light resistance (Examples 1 to 6).

In addition, it was found that the compound represented by General Formula (I) has high solubility.

On the other hand, in the comparative compound which is not the compound represented by General Formula (I), it was not possible to achieve both the high refractive index anisotropy Δn and the excellent light resistance (Comparative Examples 1 and 2).

### Example 7

As Example 7, an optical element was produced using the compound A-2 as shown below.

### [Production of Optical Element]

### <Preparation of Support and Saponification Treatment of Support>

As a support, a commercially available triacetyl cellulose film “Z-TAC” (manufactured by Fujifilm Corporation) was used.

The support was allowed to pass through a dielectric heating roll at a temperature of 60° C. so that the surface temperature of the support was increased to 40° C.

Next, an alkali solution described below was applied onto a single surface of the support using a bar coater in a coating amount of 14 mL (liter)/m², the support was heated to 110° C., and the support was transported for 10 seconds under a steam-type far infrared heater (manufactured by Noritake Co., Ltd.).

Subsequently, 3 mL/m² of pure water was applied onto the surface of the support, onto which the alkali solution had been applied, using the same bar coater. Next, water cleaning using a foundry coater and water draining using an air knife were repeated three times, and then the support was transported and dried in a drying zone at 70° C. for 10 seconds, whereby the surface of the support was subjected to the alkali saponification treatment.

Alkali solution

| | | |
|---|---|---|
| Potassium hydroxide | 4.70 | parts by mass |
| Water | 15.80 | parts by mass |
| Isopropyl alcohol | 63.70 | parts by mass |
| A surfactant SF-1: | 1.0 | part by mass |
| C₁₄H₂₉O(CH₂CH₂O)₂OH | | |
| Propylene glycol | 14.8 | parts by mass |

### <Formation of Undercoat Layer>

The following coating liquid for forming an undercoat layer was continuously applied onto the surface of the support, which had been subjected to the alkali saponification treatment, using a #8 wire bar. The support on which the coating film had been formed was dried using hot air at 60° C. for 60 seconds and further dried using hot air at 100° C. for 120 seconds to form an undercoat layer.

Coating liquid for forming undercoat layer

| | | |
|---|---|---|
| The following modified polyvinyl alcohol | 2.40 | parts by mass |
| Isopropyl alcohol | 1.60 | parts by mass |
| Methanol | 36.00 | parts by mass |
| Water | 60.00 | parts by mass |

Modified polyvinyl alcohol (the ratios of repeating units in the following structural formula is in terms of mass ratio)

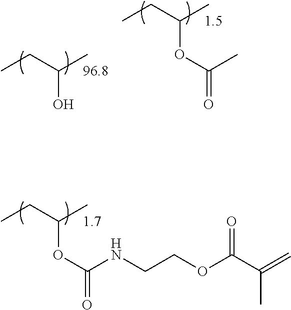

### <Formation of Alignment Film>

The following coating liquid for forming an alignment film was continuously applied onto the support, onto which the undercoat layer had been formed, using a #2 wire bar. The support on which the coating film of the coating liquid for forming an alignment film had been formed was dried using a hot plate at 60° C. for 60 seconds to form an alignment film.

Coating liquid for forming alignment film

| | | |
|---|---|---|
| A material for photo alignment | 1.00 | part by mass |
| Water | 16.00 | parts by mass |
| Butoxyethanol | 42.00 | parts by mass |
| Propylene glycol monomethyl ether | 42.00 | parts by mass |

### Material D for Photo Alignment

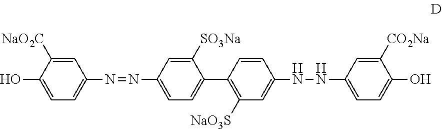

### <Exposure of Alignment Film>

An exposure film was exposed using the exposure device of FIG. 5 of WO2020/22496A to form an alignment film P-1 having an alignment pattern.

In the exposure device, a laser that emits laser light having a wavelength (325 nm) was used as the laser. The exposure amount of the interference light was 2,000 mJ/cm². It is noted that one period (the length over which the optical axis derived from the liquid crystal compound rotates 180°) of an alignment pattern formed by interference of two laser beams was controlled by changing the intersecting angle (the intersecting angle β) between the two beams.

### <Formation of Optically Anisotropic Layer>

As a composition forming the optically anisotropic layer, the following composition E-1 was prepared.

Composition E-1

| | | |
|---|---|---|
| Compound A-2 | 100.00 | part by mass |
| A polymerization initiator | 3.00 | parts by mass |
| (IRGACURE (registered trade name) | | |
| 907, manufactured by BASF SE) | | |
| The above leveling agent T-1 | 0.08 | parts by mass |
| Methyl ethyl ketone | 927.7 | parts by mass |

An optically anisotropic layer was formed by subjecting the alignment film P-1 to multilayer coating with the composition E-1. The multilayer coating refers to repeating a procedure in which, first, the composition E-1 is applied for a first layer on an alignment film, heated, and cooled, followed by being cured with ultraviolet rays to produce a liquid crystal immobilized layer, and then, for a second layer and subsequent layers, this liquid crystal immobilized layer is subjected to multiple coating by the application of the composition E-1, heating, and cooling, followed by curing with ultraviolet rays in the same manner Due to the formation by the multilayer coating, the alignment direction of the alignment film is reflected over the upper surface of the liquid crystal layer from the lower surface (the surface on the alignment film P-1 side) even in a case where the film thickness of the liquid crystal layer is increased.

First, the following composition E-1 was applied for the first liquid crystal layer onto the alignment film P-1 to form a coating film, the coating film was heated to 80° C. using a hot plate and then cooled to 50° C., followed by being irradiated with ultraviolet rays having a wavelength of 365 nm at an irradiation dose of 300 mJ/cm² using a high-pressure mercury lamp in a nitrogen atmosphere, whereby the alignment of the liquid crystal compound was fixed. At this time, the film thickness of the first liquid crystal layer was 0.3 μm.

For the second and subsequent liquid crystal layers, this liquid crystal layer was subjected to multiple coating, heating, and cooling under the same conditions as described above, followed by curing with ultraviolet rays to produce a liquid crystal immobilized layer (a cured layer). In this way, multiple coating was repeated until the in-plane retardation (Re) reached 325 nm, an optically anisotropic layer was formed, and then an optical element G-1 was produced.

Using a polarization microscope, it was confirmed that the optically anisotropic layer of this example had a periodic alignment surface as shown in FIG. 3 of WO2020/22496A. In the liquid crystal alignment pattern of the optically anisotropic layer, the one period A over which the optical axis derived from the liquid crystal compound A-2 rotated 180° was 1.0 μm. The period A was determined by measuring the period of the bright and dark pattern observed under the crossed nicol condition using a polarization microscope.

### <Measurement of Diffraction Efficiency>

An evaluation optical system in which a light source for evaluation, a polarizer, a ¼ wavelength plate, the optical element G-1, and a screen were arranged in this order was prepared. A laser pointer having a wavelength of 650 nm was used as the light source for evaluation, and SAQWPO5M-700 manufactured by Thorlabs Inc. was used as the ¼ wavelength plate. The slow axis of the ¼ wavelength plate was arranged at a relationship of 45° with respect to the absorption axis of the polarizer. In addition, the optical element G-1 was arranged so that the support surface faced the light source side.

As a result of causing the light transmitted from the light source for evaluation through the polarizer and the ¼ wavelength plate, to be incident on the optical element G-1 with being perpendicular to the film surface, a part of the light transmitted through the optical element was diffracted, and a plurality of bright spots could be confirmed on the screen.

The intensity of the diffracted light corresponding to each of the bright spots on the screen and the intensity of the zero-order light w measured with a power meter, and the diffraction efficiency was calculated according to the following expression.

Diffraction efficiency=(intensity of first-order light)/(intensity of zero-order light+intensity of diffracted light other than first-order light)

The obtained diffraction efficiency was as high as 99% or more.

### <Liquid Crystallinity of Composition>

As a result of drying the composition E-1 to volatilize the solvent (methyl ethyl ketone), it was confirmed that liquid crystallinity was exhibited.

### Example 8

As Example 8, a light guide element was produced using a composition containing the compound A-2 and a chiral agent, as shown below.

The following composition E-2 was prepared as a composition for forming a cholesteric liquid crystal layer as shown in FIG. 6 of WO2020/22496A. In the structural formula of the following chiral agent Ch-2, Bu represents an n-butyl group.

Composition E-2

| | | |
|---|---|---|
| A liquid crystal compound A-2 | 100.00 | parts by mass |
| The following polymerization initiator PI-1 | 3.00 | parts by mass |
| The following chiral agent Ch-1 | 4.40 | part by mass |
| The following chiral agent Ch-2 | 1.00 | part by mass |
| Methyl ethyl ketone | 201.31 | parts by mass |

Polymerization initiator PI-1 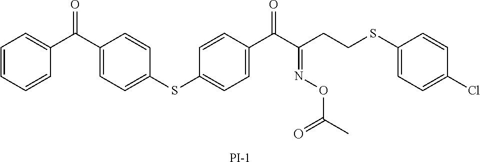 [SMILES: CC(=O)O/N=C(\CCSc1ccc(Cl)cc1)C(=O)c1ccc(Sc2ccc(C(=O)c3ccccc3)cc2)cc1 ; "PI-1"]
Chiral agent Ch-1 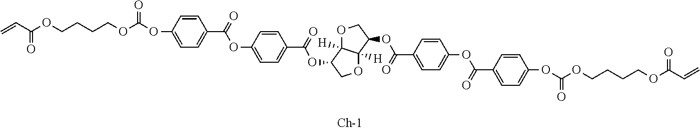 [SMILES: C=CC(=O)OCCCCOC(=O)Oc1ccc(C(=O)Oc2ccc(C(=O)O[C@H]3CO[C@H]4[C@@H]3OC[C@H]4OC(=O)c3ccc(OC(=O)c4ccc(OC(=O)OCCCCOC(=O)C=C)cc4)cc3)cc2)cc1]
Chiral agent Ch-2

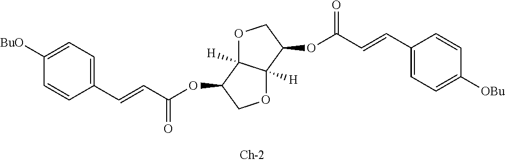

The alignment film P-1 was produced in the same manner as in the above-described <Preparation of support and saponification treatment of support>, <Formation of undercoat layer>, <Formation of alignment film>, and <Exposure of alignment film> in Example 7.

A cholesteric liquid crystal layer was formed by subjecting the alignment film P-1 to multilayer coating with the composition E-2 until the film thickness became 3.5 μm. Here, the multilayer coating refers to the repetition of the process of producing a liquid crystal immobilized layer by applying the liquid crystal composition for a first layer onto the alignment film, followed by heating and then curing with an ultraviolet ray; and then subjecting the liquid crystal immobilized layer to multiple coating for second and subsequent layers, followed by the same heating and then curing with an ultraviolet ray as described above. Due to the formation by the multilayer coating, the alignment direction of the alignment film is reflected over the upper surface of the liquid crystal layer from the lower surface even in a case where the total thickness of the liquid crystal layer is increased.

As the optically anisotropic layer for the first layer, the composition E-2 was applied onto the alignment film P-1 at 1,000 rpm using a spin coater. The coating film was heated on a hot plate at 80° C. for 3 minutes and then irradiated at 50° C. with ultraviolet rays having a wavelength of 365 nm at an irradiation dose of 300 mJ/cm² using a high-pressure mercury lamp in a nitrogen atmosphere whereby the alignment of the liquid crystal compound was fixed.

This liquid crystal layer was subjected to multiple coating for the second and subsequent liquid crystal layers, followed by heating and then curing with an ultraviolet ray under the same conditions as described above to form a cholesteric liquid crystal layer.

The formed cholesteric liquid crystal layer was bonded to a light guide plate (a glass having a refractive index of 1.80 and a thickness of 0.50 mm) to produce a light guide element.

The light having a wavelength of 532 nm was allowed to be incident in the normal direction from the light guide plate side of the produced light guide element. As a result of the above, it was confirmed that the incident light was reflected in the cholesteric liquid crystal layer beyond the critical angle in a direction different from the specular reflection direction and guided in the light guide plate.

In this way, it is possible to produce a light guide element by using the composition containing the compound represented by General Formula (I) and a chiral agent.

### <Liquid Crystallinity of Composition>

As a result of drying the composition E-2 to volatilize the solvent (methyl ethyl ketone), it was confirmed that liquid crystallinity was exhibited.

According to the present invention, it is possible to provide a compound having a high refractive index anisotropy Δn and having excellent light resistance and provide a composition, a cured product, an optically anisotropic body, an optical element, and a light guide element, each of which contains the compound.

The present invention has been described in detail and with reference to specific embodiments; however, it is apparent to those skilled in the art that various changes and modifications can be made without departing from the spirit and the scope of the invention.

What is claimed is:

1. A compound represented by General Formula (I), 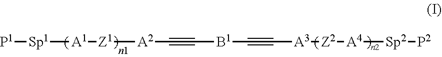
in the General Formula (I),
P¹ and P² each independently represent a hydrogen atom, —CN, —NCS, or a polymerizable group,
provided that at least one of P¹ or P² represents a polymerizable group,
Sp¹ and Sp² each independently represent an alkylene group having 1 to 15 carbon atoms, where one or more methylene groups contained in the alkylene group may be each independently replaced with —O—, —S—, or —C(═O)—,
A¹, A², A³, and A⁴ each independently represent an aromatic hydrocarbon ring group or aromatic heterocyclic group which may have a substituent L1,
in a case where a plurality of A¹'s is present, the plurality of A¹'s may be the same or different from each other,
in a case where a plurality of A⁴'s is present, the plurality of A⁴'s may be the same or different from each other,
Z¹ and Z² each independently represent —O—, —S—, —CHRCHR—, —OCHR—, —CHRO—, —CO—, —SO—, —SO₂—, —COO—, —OCO—, —CO—S—, —S—CO—, —O—CO—O—, —CO—NR—, —NR—CO—, —SCHR—, —CHRS—, —SO—CHR—, —CHR—SO—, —SO₂—CHR—, —CHR—SO₂—, —CF₂O—, —OCF₂—, —CF₂S—, —SCF₂—, —OCHRCHRO—, —SCHRCHRS—, —SO—CHRCHR—SO—, —SO₂—CHRCHR—SO₂—, —CH═CH—COO—, —C═CH—OCO—, —COO—CH═CH—, —OCO—CH═CH—, —COO—CHRCHR—, —OCO—CHRCHR—, —CR═CR—, —CR═N—, —N═CR—, —N═N—, —CR═N—N═CR—, —CF═CF—, —C≡C—, or a single bond,
R represents a hydrogen atom or an alkyl group having 1 to 10 carbon atoms,
in a case where a plurality of R's is present, the plurality of R's may be the same or different from each other,
in a case where a plurality of Z¹'s is present, the plurality of Z¹'s may be the same or different from each other,
in a case where a plurality of Z²'s is present, the plurality of Z²'s may be the same or different from each other, and
B¹ represents a group represented by any one of General Formulae (B-1) to (B-4), 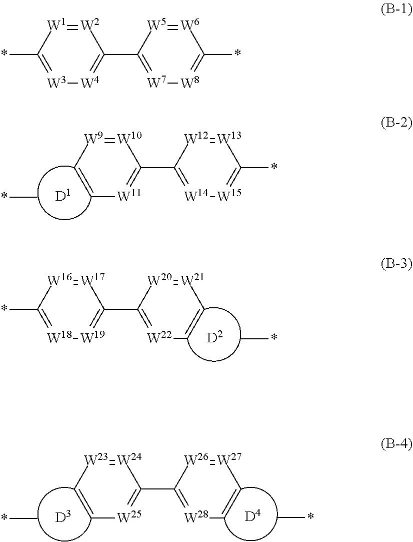
in the General Formulae (B-1) to (B-4),
W¹ to W²⁸ each independently represent CR¹ or N, where R¹ represents a hydrogen atom, an alkyl group having 1 to 10 carbon atoms, an alkoxy group having 1 to 10 carbon atoms, an alkylamino group having 1 to 10 carbon atoms, an alkylthio group having 1 to 10 carbon atoms, an alkanoyl group having 1 to 10 carbon atoms, an alkanoyloxy group having 1 to 10 carbon atoms, an alkanoylamino group having 1 to 10 carbon atoms, an alkanoylthio group having 1 to 10 carbon atoms, an alkyloxycarbonyl group having 2 to 10 carbon atoms, an alkylaminocarbonyl group having 2 to 10 carbon atoms, an alkylthiocarbonyl group having 2 to 10 carbon atoms, a hydroxy group, an amino group, a mercapto group, a carboxy group, a sulfo group, an amide group, a cyano group, a nitro group, a halogen atom, a sulfhydryl group, an aldehyde group, or a polymerizable group, provided that in a case where the group has —CH₂—, at least one —CH₂— contained in the group may be replaced with —O—, —CO—, —CH═CH—, or —C≡C—, and in a case where the group has a hydrogen atom, at least one hydrogen atom contained in the group may be replaced with at least one selected from the group consisting of a fluorine atom and a polymerizable group,
in the General Formula (B-1), at least one of W² or W⁵ represents CR¹, where R¹ represents a group other than a hydrogen atom,
at least one of W⁴ or W⁷ represents CR¹, where R¹ represents a group other than a hydrogen atom,
in the General Formula (B-2), at least one of W¹⁰ or W¹² represents CR¹, where R¹ represents a group other than a hydrogen atom,
at least one of W¹¹ or W¹⁴ represents CR¹, where R¹ represents a group other than a hydrogen atom,
in the General Formula (B-3), at least one of W¹⁷ or W²⁰ represents CR¹, where R¹ represents a group other than a hydrogen atom,
at least one of W¹⁹ or W²² represents CR¹, where R¹ represents a group other than a hydrogen atom,
in the General Formula (B-4), at least one of W²⁴ or W²⁶ represents CR¹, where R¹ represents a group other than a hydrogen atom,
at least one of W²⁵ or W²⁸ represents CR¹, where R¹ represents a group other than a hydrogen atom,
D¹, D², D³, and D⁴ each independently represent an aromatic hydrocarbon ring group or aromatic heterocyclic group which may have a substituent L2,
* represents a bonding position,
the substituent L1 and the substituent L2 each independently represent an alkyl group having 1 to 10 carbon atoms, an alkoxy group having 1 to 10 carbon atoms, an alkylamino group having 1 to 10 carbon atoms, an alkylthio group having 1 to 10 carbon atoms, an alkanoyl group having 1 to 10 carbon atoms, an alkanoyloxy group having 1 to 10 carbon atoms, an alkanoylamino group having 1 to 10 carbon atoms, an alkanoylthio group having 1 to 10 carbon atoms, an alkyloxycarbonyl group having 2 to 10 carbon atoms, an alkylaminocarbonyl group having 2 to 10 carbon atoms, an alkylthiocarbonyl group having 2 to 10 carbon atoms, a hydroxy group, an amino group, a mercapto group, a carboxy group, a sulfo group, an amide group, a cyano group, a nitro group, a halogen atom, a sulfhydryl group, an aldehyde group, or a polymerizable group, provided that in a case where the group has —CH₂—, at least one —CH₂— contained in the group may be replaced with —O—, —CO—, —CH═CH—, or —C≡C—, and in a case where the group has a hydrogen atom, at least one hydrogen atom contained in the group may be replaced with at least one selected from the group consisting of a fluorine atom and a polymerizable group, and
n1 and n2 each independently represent an integer of 0 to 2.

2. The compound according to claim 1,
wherein n1 and n2 in the General Formula (I) represent 0.

3. The compound according to claim 1,
wherein B¹ in the General Formula (I) is represented by the General Formula (B-1).

4. The compound according to claim 1,
wherein A¹, A², A³, and A⁴ in the General Formula (I) are each independently a group represented by any one of General Formulae (A-1) to (A-3), which may have the substituent L1, 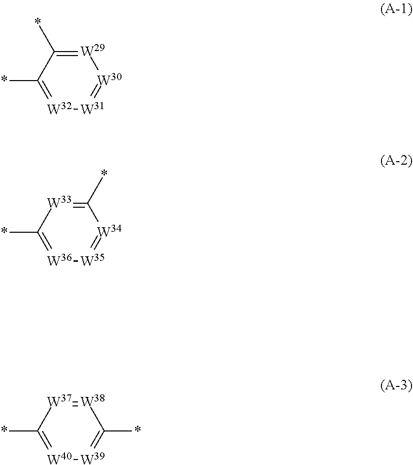
in the General Formulae (A-1) to (A-3),
W²⁹ to W⁴⁰ each independently represent CR¹ or N, where R¹ represents a hydrogen atom or the substituent L1, and
* represents a bonding position.

5. The compound according to claim 1,
wherein in the General Formula (I), Sp¹ represents a group represented by General Formula (II), and Sp² represents a group represented by General Formula (III),
*—S—W⁴¹—** (II)
*—S—W⁴²—** (III)
in the General Formulae (II) and (III),
W⁴¹ and W⁴² each independently represent an alkylene group having 1 to 15 carbon atoms, where one or more methylene groups contained in the alkylene group may be each independently replaced with —O—, —S—, or —C(═O)—,
*'s each represent a bonding position to A¹, A², A³, or A⁴, which is directly linked to Sp¹ or Sp², and
**'s each represent a bonding position to P¹ or P².

6. The compound according to claim 1,
wherein the compound has liquid crystallinity.

7. A composition comprising:
the compound according to claim 1.

8. The composition according to claim 7, further comprising:
a polymerization initiator.

9. The composition according to claim 7, further comprising:
a chiral agent.

10. The composition according to claim 7,
wherein the composition has liquid crystallinity.

11. The composition according to claim 7,
wherein the composition is used for forming an optically anisotropic layer.

12. A cured product that is obtained by curing the composition according to claim 7.

13. An optically anisotropic body that is obtained by curing the composition according to claim 7.

14. An optical element comprising:
an optically anisotropic layer formed from the composition according to claim 7,
wherein the optically anisotropic layer has an alignment pattern, and
the alignment pattern is an alignment pattern in which an orientation of an optical axis, derived from a compound contained in the composition, continuously changes rotationally along at least one in-plane direction.

15. A light guide element comprising:
the optical element according to claim 14; and
a light guide plate.
